(19) United States
(12) Patent Application Publication
(10) Pub. No.: US 2012/0149587 A1
(43) Pub. Date: Jun. 14, 2012

(54) METHOD FOR DETECTING NUCLEATED CELLS

(75) Inventors: James P. Landers, Charlottesville, VA (US); Jingyi Li, Charlottesville, VA (US); Daniel C. Leslie, Brookline, MA (US)

(73) Assignee: University of Virginia Patent Foundation, Charlottesville, VA (US)

(21) Appl. No.: 13/116,659

(22) Filed: May 26, 2011

Related U.S. Application Data
(63) Continuation-in-part of application No. 12/879,810, filed on Sep. 10, 2010.
Application No. 12/879,810 is a continuation of application No. PCT/US2009/036983, filed on Mar. 12, 2009.
(60) Provisional application No. 61/035,923, filed on Mar. 12, 2008.

(51) Int. Cl. C40B 30/00; C12Q 1/68

(52) U.S. Cl. 506/7; 435/6.1; 435/6.12

(57) ABSTRACT

The invention provides methods to detect or quantify cells such as nucleated cells in a sample such as a physiological sample, which employ magnetic substrates and subjects the sample and the magnetic substrate to forms of energy so as to induce aggregate formation.

### CROSS-REFERENCE TO RELATED APPLICATIONS

This application is a continuation in part of U.S. application Ser. No. 12/879,810, filed on Sep. 10, 2010, which is a continuation under 35 U.S.C. §111(a) of International Patent Application Serial No. PCT/US2009/036983, filed on Mar. 12, 2009, which claims the benefit of the filing date of U.S. application Ser. No. 61/035,923, filed on Mar. 12, 2008, and also claims the benefit of the filing date of PCT/US2010/02883, filed on Nov. 3, 2010, which claims the benefit of the filing date of U.S. application Ser. No. 61/257,679, filed on Nov. 3, 2009, and U.S. application Ser. No. 61/384,534, filed on Sep. 20, 2010, the disclosures of which are incorporated by reference herein.

### BACKGROUND

Polymeric analytes can be detected using methods, such as chromatography, electrophoresis, binding assays, spectrophotometry, and the like. DNA detection, for instance, may require expensive, bulky optics for either absorbance-based techniques or intercalating-dye fluorescence based techniques. Although DNA concentration has routinely been detected spectrometrically by measuring absorbance ratio of a sample at 260/280 nm, the method suffers from poor sensitivity at low concentrations of DNA.

Other methods for DNA detection include DNA binding to a fluorescence dye and detecting the fluorescence using a fluorometer. Examples of such a dye are PicoGreen®, which is commercially available through Invitrogen (Carlsbad, Calif.) (see Ahn et al., Nucl. Acids Res., 24:2623 (1996); Vitzthum et al., Anal. Biochem., 276:59 (1999), and dyes disclosed in U.S. Pat. Nos. 6,664,047; 5,582,977 and 5,321,130. Additional DNA quantification methods based on fluorescence have been developed and include oligortucleotide hybridization (Sanchez et al., J. Clin. Microbiol., 40:2381 (2002)) and real-time quantitative PCR (Heid et al., Genome Res., 6:986 (1996)). While highly sensitive, fluorometer-based methods are generally cumbersome, requiring reagent preparation and handling and a special fluorometer for exciting and measuring fluoro-emission.

Hague et al. (BMC Biotech., 3:20 (2003)) compared three popular DNA quantification methods with regard to accuracy: OD₂₆₀/OD₂₈₀ (OD), PicoGreen® double stranded DNA (PG), and detection of fluorescent signal from a 5′ exonuclease assay (quantitative genomic method (QG), based on the TaqMan® assay). Their exhaustive analysis, involving nearly 15,000 measurements, revealed that OD measurement was the most precise and least biased method for estimating DNA concentration. Among the benefits of that method are the relatively wide availability of absorbance spectrophotometers in contrast to fluorometers, that OD measurement does not consume sample or additional reagents, and that no time is required for incubation or reaction time, as is the case with a fluorophore. On the other hand, a large amount of sample is needed for OD measurement, and this method does not discriminate between single stranded and double stranded DNA (as PG does) (Singer et al., Anal. Biochem., 249:228 (1997)) or contaminating DNA (as the sequence specific QG method. does). In addition, the presence of protein, RNA and salt can lead to an overestimate of DNA concentration from OD measurements.

Among the benefits of fluorometric methods are the use of very small sample volumes due to the high sensitivity of the methods and that fluorescence detection is easily implemented in microdevices. However, some reagents are not compatible with fluorescence based DNA quantification due to signal quenching.

### SUMMARY OF THE INVENTION

The invention provides label-free detection technology based on solid substrate, e.g., magnetic particle, for instance magnetic bead, aggregation, in the presence of polymeric molecules, such as nucleic acid containing cells, such as nucleated (eukaryotic) cells and other cellular molecules found in complex biological samples, and an energy source such as a rotating magnetic field (RMF), pulsating heat, acoustic energy or mechanical agitation. The aggregation, for instance, the formation of pinwheel shaped structures, can be visually detected and/or quantified. Moreover, the opaque nature of the aggregated particles makes the transition very easy to monitor optically, and simple image analysis techniques can be used to extract quantitative information. The combination of high sensitivity and simplicity of the method provides in one embodiment a label-free approach to DNA or RNA detection and/or quantification, and thereby nucleic acid containing cell quantification. The observable effect for nucleic acid is also quite robust even in the presence of proteins and lipids at concentrations typically encountered in biological samples. The methods of the invention have specific advantages for automated assays in microfluidic platforms.

Thus, aggregate, e.g., pinwheel, formation may be detected visually, which requires minimal footprint or expensive optical equipment, and can be employed to quantify the amount of many different polymeric analytes in a sample, such as a complex biological sample having protein, carbohydrates such as polysaccharides, nucleic acid, and/or lipid, or any combination thereof. Aggregate formation may be detected using microscopy, photography, scanners, magnetic sensing and the like.

For example, the stark differences in optical contrast of images in the absence and presence of DNA allows for the use simple digital image processing to define a quantitative relationship between the mass of DNA and the extent of particle (e.g., bead) aggregation. This relationship was determined via an algorithm based on the gray value of the digital image. A threshold gray level is set such that dispersed beads and clusters are counted as “dark,” whereas areas in the image cleared of beads are counted as “bright.” The number of dark pixels in the image is then used as a measure of aggregation, with 100% dark area representing a sample without aggregation, whereas low dark area percentages correspond to nearly complete aggregation. In one embodiment, the dark area percentage decreased with increasing DNA concentration up to about 80 pg/μL, with a limit of detection of better than 1 pg/μL. Even without optimization, the dynamic range of pinwheel formation as a metric for DNA quantitation covered about 2.5 orders of magnitude.

In one embodiment, under concentrated chaotropic salt conditions, e.g., salts such as guanidine hydrochloride, guanidine thiocyanate, ammonium perchlorate and the like, the formation of pinwheels is specific for the presence of DNA and/or RNA (nucleic acid) in a cellular sample and that formation is not inhibited by the presence of protein or other cellular components, even at concentrations that greatly exceed that of the nucleic acid, e.g., DNA. For example, readily detectable aggregates occur at concentrations as low as 0.3 pg/μL, of DNA in a complex sample.

As discussed herein, pinwheel formation is effective with samples other than prepurified DNA. The same effect is observed for cultured mouse cells pipetted directly into a guanidine HCl-bead solution and for complex samples like human whole blood. The mass of protein in whole blood ranges from about 60 to about 83 mg/μL and dwarfs the mass of DNA (about 25 to about 63 ng/μL, assuming 6.25 pg/white blood cell (WBC), and about 4,000 to 10,000 WBCs/μL). Crude separation of the components of whole blood can be achieved with a benchtop centrifuge into plasma (cell-free component), buffy coat (white cells) and red cells. Aliquots from the buffy coat strongly induced the pinwheel effect and pinwheel formation was associated with the fraction having DNA containing cells. Even though aggregation may be limited, it was nonetheless readily detected: detection of DNA at levels of about 0.3 pg/μL was robust amidst an overwhelming concentration of protein (about 70 mg/μL). Although plasma DNA concentration is typically about 10 to about 50 pg/μL, the fragment lengths of that DNA are typically <1000 bp which may not allow for substantial pinwheel formation for this bead size, c.a., beads of about 1 to 8 micrometers. However, smaller bead sizes (sub 1 micrometer) may increase sensitivity for shorter nucleic acid fragment lengths.

Thus, the quantitative behavior of particle, e.g., bead, aggregation can be used to directly determine the concentration of nucleated cells in whole blood. Based on the average mass of DNA contained within a WBC, the DNA quantitation values obtained from the pinwheel results can be used to back-calculate the number of WBC in whole blood samples. Moreover, the results obtained with the simple pinwheel assay (in less than 5 minutes; average of triplicate analysis) yielded results that were comparable to those obtained by the more sophisticated Coulter counting method.

In one embodiment, pinwheel formation was observed down to 30 pg of DNA (prepurified or in cells). In one embodiment, pinwheel formation was observed down to 150 fM of DNA. In one embodiment, concentrations as low as about 3 to about 10 pg/μL of nucleic acid in a sample may be detected using a pinwheel assay. In one embodiment, pinwheel formation may not be observed if high molecular weight DNA is sonicated into smaller fragments, fragments of less than about 5,000 to about 10,000 base pairs or about a few hundred base pairs in length, for instance, less than about 900, 700, 500, 300, or 200 base pairs in length, using about 4 to about 12 micrometer (micron) diameter particles. However, smaller particles, e.g., beads, of about 5 microns (μm) may be useful in detecting and/or quantitating nucleic acid of about 1,000 to about 5,000 base pairs in length.

Thus, the invention provides a method for detecting the presence or amount of a nucleic acid analyte in a complex biological sample. The method includes contacting the complex biological sample with magnetic beads, e.g., from about 1 nm to about 300 micrometers in diameter, under conditions that allow for binding of the analyte to the beads so as to form a mixture. In one embodiment, the beads include a paramagnetic metal. The mixture is subjected to energy, e.g., a rotating magnetic field or acoustic energy, and the presence or amount of pinwheels or aggregates in the mixture is detected or determined. In one embodiment, the mixture is contacted with a magnet which induces pinwheel or aggregate formation. In one embodiment, pinwheels or aggregates are isolated from the mixture, thereby isolating the analyte. For example, the pinwheels or aggregates may be magnetically isolated. In one embodiment, after pinwheel or aggregate formation is detected or determined, in the absence of contact with a magnet or the rotating magnetic field (e.g., the field is turned off) or other applied energy, the aqueous solution in the mixture having the pinwheels or aggregates is removed and an elution buffer is added to form a second mixture having the pinwheels or aggregates. In one embodiment, the second mixture is subjected to the rotating magnetic field or other applied energy.

In one embodiment, the method for detecting the presence or amount of a polymeric analyte in a sample employs magnetic beads but not a rotating magnetic field. In this embodiment, a sample having a polymeric analyte and magnetic beads are subjected to other forms of energy, e.g., vibration such as that from a speaker (acoustic energy), so as to form aggregates. In one embodiment, the sample is a complex biological sample. Aggregate formation is then detected or determined.

Thus, the invention provides a quantitative method. Unlike methods that purify an analyte, such as DNA, before quantitation, methods described herein allow for quantitation without prior purification.

In one embodiment, the invention provides a method for detecting the presence or amount of a nucleic acid analyte in a complex biological sample. The method includes contacting the complex biological sample with magnetic beads under conditions that allow for binding of the nucleic acid analyte to the beads so as to form a mixture. The mixture is subjecting to a rotating magnetic field, a magnet or other applied energy and the presence or amount of pinwheels or aggregates in the mixture is detected, thereby detecting the presence or amount of the analyte in the sample.

Also provided is a method to isolate an analyte, e.g., from a complex sample. The method includes contacting the sample with magnetic beads in a solution, such as an aqueous solution, under conditions that allow for binding of the analyte to the beads so as to forma mixture. The mixture is subjecting to a rotating magnetic field, a magnet or other applied energy that results in aggregation of the beads having the bound analyte but not other molecules in the complex sample. For example, for a cellular sample where nucleic acid is the analyte for isolation, aggregation of the beads isolates the nucleic acid from other cellular components such as proteins, lipids, carbohydrates and the like. The cellular debris can be removed by removing the solution from the aggregate containing mixture and the nucleic acid can be eluted by adding a buffer, e.g., a Tris-EDTA containing buffer, to the aggregates, and the analyte containing buffer collected.

In one embodiment, the invention provides a method to determine the specific amount of an analyte in a solution using magnetic beads, e.g., silica-coated magnetic beads. This may be accomplished with a camera and routine image processing software. The method may be applied to quantifying nucleic acids undergoing amplification, e.g., rolling circle amplification and whole genome amplification, where the products have higher molecular weights than products produced using some other nucleic amplification methods, such as the polymerase chain reaction. In one embodiment, the method is sensitive to about 20 human cells in 20 microliters of solution. The quantification method may also be applied to non-nucleic acid polymeric analytes, such as the polysaccharide chitosan, where a dose-dependent aggregation was also observed in a similar manner to the DNA induced pinwheel formation on beads under non-chaotropic conditions. Under these conditions, the negatively charged silica bead surface is electrostatically attracted to the cationic chitosan (protonated amine) under low ionic strength conditions at physiological pH. The method may be altered to include fluorescently labeled magnetic beads or measurements of the magnetic susceptibility of the aggregates, to increase the sensitivity of the assay. Moreover, the method may be employed as a step in the purification of molecules bound to the beads, e.g., nucleic acids.

The invention also provides methods of using particles which employ sequence specific oligonucleotides. For example, beads having a ferromagnetic core and a polystyrene shell were linked to one of two different oligonucleotides through biotinylation, resulting in two types of beads. The two different single-stranded oligonucleotides were complementary to the 3′ and 5′ ends of a target sequence (the ‘connector’). To demonstrate temperature-dependent, hybridization-induced aggregation, a 500 base pair fragment from the 48.5 kbp genome of the lambda phage was used as a target sequence. The oligonucleotides (e.g., those used as primers in PCR amplification of the target) were biotinylated to magnetic 1 μm beads. Sequence-specific aggregation of the 500 bp λ DNA target with its complimentary bead-bound oligonucleotides was found to be temperature-dependent (the annealing temperature for hybridization of the 500 base target to the λ3′- and 5′-oligonucleotides was about 70° C.). In one embodiment, the connector and complementary oligonucleotides may be selected from a proximity ligation assay in order to ensure hybridization, and thus aggregation, at room temperature in a sequence-specific manner. In the absence of the connector, the beads disperse homogeneously in a buffer solution.

Moreover, the connector, which is a sequence to be detected in a sample, may be fairly short. For instance, addition of 6×10¹⁰ copies (corresponding to a concentration of about 100 nM or about 8000 pg/μL) of a 26-base connector to a bead suspension immediately led to bead aggregation in the presence of a RMF. Aggregation was observed at concentrations as low as 1 fM (corresponding to only 600 copies of the connector, or 0.000079 pg/μL). The specificity of the aggregation was shown by using a connector with. near similar base composition to the 26-base connector that induced aggregation via hybridization, but was not complementary to the oligonucleotides on the beads. No aggregation was observed even at the extremely high concentration of 100 nM (about 6×10¹⁰ copies, or about 7900 pg/μL). Fluorescence assays based on intercalating dyes have a detection threshold around 6×10⁵ copies (1 pM, or 0.079 pg/μL). By contrast, hybridization induced aggregation could clearly be detected at concentrations three orders of magnitude lower, e.g., 0.000079 pg/μL. Quantitative information can be extracted from such assays by image analysis. Moreover, the response for many applications need only be digital.

Thus, the invention also provides a hybridization induced aggregation assay, a homogenous assay. Unlike inducing pinwheel formation with high molecular weight (long molecules) of DNA under chaotropic conditions, the invention also provides for the detection and/or quantification of sequence-specific DNA (or other nucleic acid of appropriate length) via pinwheel formation, for instance, under physiological conditions. The magnetic beads (or other magnetic substrates) employed in one embodiment of the hybridization-induced aggregation assay include oligonucleotides specific for a target nucleic acid sequence. Pairs of oligonucleotides bound to beads, via non-covalent interactions, aggregate when ‘connector’ (target) sequences are present. The use of non-covalent interactions may allow for easier coupling and post-pinwheel release of target sequences and/or oligonucleotides. The length of a target nucleic acid sequence can be as short as 10 bases to as long as hundreds of millions of bases in length with a binding sequence of 4 bases on each end with sequences in the bead bound oligonucleotides. A mixture with the beads and the target nucleic acid sequence, when heated to an appropriate temperature (annealing T), results in hybridized (annealed) sequences, which subsequently induce aggregation. Although sequence-specific induced pinwheeling can be used to detect target sequences in long molecules of DNA, e.g., genomic DNA, efficient hybridization induced aggregation occurs with shorter target nucleic acid molecules and under non-chaotropic conditions. To provide for shorter fragments of high molecular weight nucleic acids (intact cellular DNA), hydrodynamic shear forces are used to cause covalent bond breakage. Simply mixing, pouring, pipetting, or centrifuging DNA containing solutions, or subjecting high molecular weight DNA to sonication or shearing through a needle or nuclease treatment, may generate shorter fragments.

The hybridization based assay is particularly useful to detect markers including, but are not limited to, cancer markers, genetically-modified food, genetically-modified organisms, human genomic markers (relative to other DNA), or bacterial genome markers. The homogenous assay may contain a series of the same type of beads with different oligonucleotides, where each pair of beads has sequences specific for a different target sequence having a different annealing temperature, or may have beads with different properties (such as in size or surface chemistry) that allow for distinguishing the presence of different target sequences in a sample. In one embodiment, the detection of pinwheeling at select temperature (T) as the sample traverses a temperature range of annealing T, allows for the detection of the presence of certain DNA sequences.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. A REMF (A) centered on a microfluidic chamber containing a minute mass of magnetic silica beads (B, white dotted line), reveals the presence of a select polymeric analyte in the sample through bead aggregation and the formation of ‘pinwheels’ (C). When the sample is devoid of specific polymeric analytes, the beads remain in the ‘dispersed’ formation (D). [A,B-photographs; CD-micrographs at 20 times magnification]. The effect was originally reported with genomic DNA but could not confirm specificity in the presence of protein.

FIG. 2. 5 μm magnetic beads in a rotating magnetic field with buffer (A), with 15 ng of human genomic DNA (B), with 1 mg/mL BSA (C), and with both 15 ng of DNA and 1 mg/mL BSA in a chaotropic, high salt solution (D).

FIG. 3. 5 μm magnetic beads in a rotating magnetic field with 30 ng (A), 15 ng (B), 3 ng (C), 300 pg (D), and 30 pg (E) of human genomic DNA with 4, 2, 1, 0.2, and 0.2 μL of beads, respectively in a chaotropic, high salt solution.

FIG. 4. 5 μm magnetic beads in a rotating magnetic field with 40 ng of DNA before sonication (A), with 10 ng of DNA after sonication (B), and without any DNA in a chaotropic, high salt solution.

FIG. 5. 5 μm magnetic beads in a rotating magnetic field with a low salt buffer (A), with 20 ng of chitosan, a multiply positively charged polysaccharide, in a low salt buffer (B), and with 20 ng of DNA in a high salt chaotropic buffer (C).

FIG. 6. (A) Graph of the percent dark area (pixels) versus mass of DNA in samples with pinwheels shown in photographs of wells (B). The sample and silica-coated superparamagnetic beads (Magnesil™) in 6-8 M guanidine hydrochloride solution, which forces the nucleic acids (NA) onto the beads surface, were mixed. The photographs (5 for each data point, error bars denote 1 standard deviation) were analyzed with imageJ software (http://rsbweb.nih.gov/ij/) to quantify the dark pixels (area of beads) in the well exposure (A). Samples were normalized to the value of dark area in the negative control and expressed as a percentage of dark area. The assay is shown to be reproducible over multiple samples (C).

FIG. 7. Pinwheel formation is not unique to DNA in a chaotropic solution. Chitosan, a cationic polysaccharide (MW about 310 kDa), forms distinct pinwheels with the same silica beads in a low-salt buffer (A to F, increasing polymer). The binding of chitosan to the beads is governed by electrostatic attraction, demonstrating that this detection method can be extrapolated to a wide variety of polymeric analytes with different binding chemistries.

FIG. 8. The sensitivity of the assay is shown to be a function of the amount of beads in relation to the amount of DNA (A). The sensitivity of the assay decreased with increasing amounts of beads. The assay with the largest amount of beads was replotted with a linear fit (B) with a 0.9869 R² value.

FIG. 9. The pinwheel effect was observed in an assay of a clinical sample of human blood treated with EDTA (anti-coagulant) (B). The image analysis revealed a logarithmic signal magnitude with increasing blood volume (A). Indicative of the DNA mechanism, the pinwheel effect was observed primarily in the huffy coat portion of a centrifuged sample of blood, regardless of plasma addition, but was not observed in pure plasma (C).

FIG. 10. Graph of the number of pixels versus grey level. The grey level is set by software so that there is a maximum distance below the threshold using the triangle algorithm.

FIG. 11. HeLa cells were mixed with MagnaSil™ paramagnetic particles and imaging used to determine the normalized percent of dark area in the sample.

FIG. 12. (A) Schematic of hybridization induced aggregation and exemplary oligonucleotides and target sequences, SEQ ID NO: 1-3 and 10 are shown. (B) The effect of altering amount of connector in the hybridization induced aggregation assay.

FIG. 13. Detection of a PCR product using hybridization induced aggregation. SEQ ID NO: 4 & 5 are shown.

FIG. 14. (A) Photograph of a blood sample analyzed by the pinwheel assay. The pinwheel results are given as an average (±SD) for an n=3 with image processing involving 5 photographs shot over 30 seconds. The pictures are analyzed using ImageJ v1.41. For each picture., a threshold, value is set automatically by isodata algorithm, which defines pixels representing the particles, and then the number of these pixels is counted. (B) A graph of the percent of the dark area. Values are normalized by that of a negative control (no DNA) as a function of the amount of DNA or cell.

FIG. 15. Blood samples analyzed by the pinwheel assay and by Coulter Counter cell count. The pinwheel results are given as an average (±SD) for an n=3 with image processing involving 5 photographs shot over 30 seconds. (A) Bar graph of white blood cells (WBCs) per μL in three samples detected by the two methods. (B) The pinwheel effect can be utilized to determine the concentration of DNA directly from blood samples. Using normalized percentage of ‘dark’ pixels with constant volume (3.5 nL) the comparison of three different human blood samples was accomplished. Different concentrations for each of the samples correlated with measurement of DNA via the conventional method (panel A). The results are displayed as the normalized percentage of ‘dark’ pixels with increasing amount of human blood (scaled by DNA amount).

FIG. 16. Direct comparison of results for WBC counts using the pinwheel assay versus a Coulter Counter.

FIG. 17. Three dimensional schematic of WBC/μL versus dark area versus the dilution factor.

FIG. 18 shows a two dimensional version of FIG. 17.

### DETAILED DESCRIPTION OF THE INVENTION

### Definitions

A “detectable moiety” is a label molecule attached to, or synthesized as part of, a solid substrate for use in the methods of the invention. These detectable moieties include but are not limited to radioisotopes, colorimetric, fluorometric or chemiluminescent molecules, enzymes, haptens, redox-active electron transfer moieties such as transition metal complexes, metal labels such as silver or gold particles, or even unique oligonucleotide sequences.

As used herein, the terms “label” refers to a marker that may be detected by photonic, electronic, opto-electronic, magnetic, gravimetric, acoustic, enzymatic, magnetic, paramagnetic, or other physical or chemical means. The term “labeled” refers to incorporation of such a marker, e.g., by incorporation of a radiolabeled molecule or attachment to a solid substrate that may be suspended in solution such as a bead.

A “biological sample” can be obtained from an organism, e.g., it can be a physiological fluid or tissue sample, such as one from a human patient, a laboratory mammal such as a mouse, rat, pig, monkey or other member of the primate family, by drawing a blood sample, sputum sample, spinal fluid sample, a urine sample, a rectal swab, a peri-rectal swab, a nasal swab, a throat swab, or a culture of such a sample, or from a plant or a culture of plant cells. Thus, biological samples include, but are not limited to, whole blood or components thereof, blood or components thereof, blood or components thereof, semen, cell lysates, saliva, tears, urine, fecal material, sweat, buccal, skin, cerebrospinal fluid, and hair. In one embodiment, the biological sample comprises cells.

“Analyte” or “target analyte” is a substance to be detected in a biological sample such as a physiological sample using the present invention. “Polymeric analyte” as used herein refers to macromolecules that are made up of repeating structural units that may or may not be identical. The polymeric analyte can include biopolymers or non-biopolymers. Biopolymers include, but are not limited to, nucleic acids (such as DNA or RNA), proteins, polypeptides, polysaccharides (such as starch, glycogen, cellulose, or chitin), and lipids

“Capture moiety” is a specific binding member, capable of binding another molecule (a ligand), which moiety or its ligand may be directly or indirectly attached through covalent or noncovalent interactions to a substrate (bead). When the interaction of the two species produces a non-covalently bound complex, the binding which occurs may be the result of electrostatic interactions, hydrogen-bonding, or lipophilic interactions. The term “ligand” refers to any organic compound for which a receptor or other binding molecule naturally exists or can be prepared. Binding pairs useful as capture moieties and ligands include, but are not limited to, complementary nucleic acid sequences capable of forming a stable hybrid under suitable conditions, antibodies and the ligands therefore, enzymes and substrates therefore, receptors and agonists therefore, lectins and carbohydrates, avidin and biotin, streptavidin and biotin, and combinations thereof. In one embodiment, the affinity of a capture moiety and its ligand may be greater than about 10⁻⁵ M, such as greater than about 10⁻⁶ M, including greater than about 10⁻⁸ M and greater than about 10⁻⁹ M. In embodiment, oligonucleotides having biotin labels are bound to beads coupled to streptavidin.

The term “homology” refers to sequence similarity between two nucleic acid molecules. Homology may be determined by comparing a position in each sequence, which may be aligned for purposes of comparison. When a position in the compared sequence is occupied by the same base, then the molecules are homologous at that position. A degree of homology between sequences is a function of the number of matching or homologous positions shared by the sequences.

“Identity” means the degree of sequence relatedness between polynucleotide sequences, as the case may be, as determined by the match between strings of such sequences. “Identity” and “homology” can be readily calculated by known methods. Suitable computer program methods to determine identity and homology between two sequences include, but are not limited to, the GCG program package (Devereux, et al., Nucleic Acids Research, 12:387 (1984)), BLASTN, and, FASTA (Atschul et al., J. Molec. Biol., 215:403 (1990)). The BLAST X program is publicly available from NCBI and other sources (BLAST Manual, Altschul et al., NCBI NLM NIH Bethesda, Md. 20894; Altschul et al., J. Mol. Biol., 215:403 (1990)).

As used, herein, the term “amount” is intended to mean the level of a molecule. The term can be used to refer to an absolute amount of a molecule in a sample or relative to a control molecule. For example, when detecting specific sequences, a reference or control amount may be a normal reference level or a disease-state reference level. A normal reference level may be an amount of expression of a biomarker in a non-diseased subject or subjects. A disease-state reference level may be an amount of expression of a biomarker in a subject with a positive diagnosis for the disease or condition.

As used herein, the term “subject” means the subject is a mammal, such as a human, but can also be an animal, e.g., domestic animals (e.g., dogs, cats and the like), farm animals (e.g., cows, sheep, pigs, horses and the like) and laboratory.

A “paramagnetic metal” is a metal with unpaired electrons. Suitable paramagnetic metals include transition elements and lanthanide series inner transition elements. Additional suitable paramagnetic metals include, e.g., Yttrium (Y), Molybdenum (Mo), Technetium (Tc), Ruthenium (Ru), Rhodium (Rh), Tungsten (W), and Gold (Au). Additional specific suitable specific paramagnetic metals include, e.g., Y(III), Mo(VI), Tc(IV), Tc(VI), Tc(VII), Ru(III), Rh(III), W(VI), Au(I), and Au(III).

A lanthanide,” “lanthanide series element” or “lanthanide series inner transition element” refers to Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), or Lutetium (Lu). Specific suitable lanthanides include, e.g., Ce(III), Ce(IV), Pr(III), Nd(III), Pm(III), Sm(II), Sm(III), Eu(II), Eu(III), Gd(III), Tb(III), Dy(III), Ho(III), Er(III), Tm(III), Yb(II), Yb(III), and Lu(III).

Examples of transition metal oxides include, but are not limited to: CrO₂, COFe₂O₄, CuFe₂O₄, Dy₃Fe₅O₁₂, DyFeO₃, ErFeO₃, Fe₅Gd₃O₁₂, Fe₅HO₃O₁₂, FeMnNiO₄, Fe₂O₃, γ-Fe₃O₄ (magnetite), α-Fe₃O₄ (hematite), FeLaO₃, MgFe₂O₄, Fe₂MnO₄, MnO₂, Nd₂O₇Ti₂, Al₀2Fe₁8NiO₄, Fe₂Ni_{0.5}O₄Zn_{0.5}, Fe₂Ni_{0.4}Zn_{0.6}, Fe₂Ni_{0.8}Zn_{0.2}, NiO, Fe₂NiO₄, Fe₅O₁₂Sm₃, Ag_{0.5}Fe₁₂La_{0.5}O₁₉, Fe₅O₁₂Y₃, and FeO₃Y. Oxides of two or more of the following metal ions can also be used: Al(+3), Ti(+4), V(+3), Mn(+2), Co(+2), Ni(+2), Mo(+5), Pd(+3), Ag(+1), Cd(+2), Gd(+3), Tb(+3), Dy(+3), Er(+3), Tm(+3) and Hg(+1).

As used herein, a “nucleic acid sequence,” a “nucleic acid molecule” or “nucleic acids” refers to one or more ofigonucleotides or polynucleotides as defined herein. As used herein, a “target nucleic acid molecule” or “target nucleic acid sequence” refers to an oligonucleotide or polynucleotide comprising a sequence that a user of a method of the invention desires to detect in a sample.

The term “polynucleotide” as referred to herein means a single-stranded or double-stranded nucleic acid polymer composed of multiple nucleotides. In certain embodiments, the nucleotides comprising the polynucleotide can be ribonucleotides or deoxyribonueleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate, and phosphoroamidate. The term “polynucleotide” specifically includes single and double stranded forms of DNA.

The term “oligonucleotide” referred to herein includes naturally occurring, and modified nucleotides linked together by naturally occurring, and/or non-naturally occurring oligonucleotide linkages. Oligonucleotides are a polynucleotide subset comprising members that are generally single-stranded and have a length of 200 bases or fewer. In certain embodiments, oligonucleotides are 2 to 60 bases in length. In certain embodiments, oligonucleotides are 12, 13, 14, 15, 16, 17, 18, 19, 20, 21, 22, 23, 24, or 25 to 40 bases in length. In certain other embodiments, oligonucleotides are 25 or fewer bases in length. Oligonucleotides of the invention may be sense or antisense oligonucleotides with reference to a protein-coding sequence.

The term “naturally occurring nucleotides” includes deoxyribonucleotides and ribonucleolides, The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphomamidate, and the like. See, e.g., LaPlanche et al., Nucl. Acids Res., 14:9081 (1986); Stec et al., J. Am. Chem. Soc., 106:6077 (1984); Stein et al., Nucl. Acids Res., 16:3209 (1988); Zon et al., Anti-Cancer Drug Design, 6:539 (1991); Zon et al., OLIGONUCLEOTIDES AND ANALOGUES: A PRACTICAL APPROACH, pp. 87-108 (F. Eckstein, Ed.), Oxford University Press, Oxford England (1991); U.S. Pat. No. 5,151,510; Uhlmann and Peyman, Chemical Reviews, 90:543 (1990), the disclosures of which are hereby incorporated by reference for any purpose. An oligonucleotide can include a detectable label to enable detection of the oligonucleotide or hybridization thereof.

The term “highly stringent conditions” refers to those conditions that are designed to permit hybridization of nucleic acid strands whose sequences are highly complementary, and to exclude hybridization of significantly mismatched sequences. Hybridization stringency is principally determined by temperature, ionic strength, and the concentration of denaturing agents such formamide. Examples of “highly stringent conditions” for solution without bead aggregation) hybridization and washing are 0.015 M sodium chloride, 0.0015 M sodium citrate at 65-68° C. or 0.015 M sodium chloride, 0.0015 M sodium citrate, and 50% formamide 42° C. See Sambrook, Fritsch & Martians, Molecular Cloning: A Laboratory Manual (2nd ed., Cold Spring Harbor Laboratory, 1989); Anderson et al., Nucleic Acid Hybridisation: A Practical Approach Ch. 4 (IRL, Press Limited).

More stringent conditions (such as higher temperature, lower ionic strength, higher formamide, or other denaturing agent) may also be used—however, the rate of hybridization will be affected. Other agents may be included in the solution hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO₄, (SDS), ficoll, Denhardt's solution, sonicated salmon sperm DNA (or another non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4; however, at typical ionic strength conditions, the rate of hybridization is nearly independent of pH. See Anderson et al., Nucleic Acid Hybridisation: A Practical Approach Ch. 4 (IRL Press Limited).

Factors affecting the stability of duplexes include base composition, length, and degree of base pair mismatch. Hybridization conditions can be adjusted by one skilled in the art in order to accommodate these variables and allow nucleic acids of different sequence relatedness to form hybrids. For example, the melting temperature of a perfectly matched DNA duplex can be estimated by the following equation: Tₘ(° C.)=81.5+16.6(log[Na+])+0.41(% G+C)-600/N-0.72(% formamide) where N is the length of the duplex formed, [Na+] is the molar concentration of the sodium ion in the hybridization or washing solution, % G+C is the percentage of (guanine±cytosine) bases in the hybrid. For imperfectly matched hybrids, the melting temperature is reduced by approximately 1° C. for each 1% mismatch.

The term “moderately stringent conditions” refers to conditions under which a duplex with a greater degree of base pair mismatching than could occur under “highly stringent conditions” is able to form. Examples of typical “moderately stringent conditions” in solution are 0.015 M sodium chloride, 0.0015 M sodium citrate at 50-65° C. or 0.015 M sodium chloride, 0.0015 M sodium citrate, and 20% formamide at 37-50° C. By way of example, “moderately stringent conditions” of 50 degree C. in 0.015 M sodium ion will allow about a 21% mismatch.

It will be appreciated by those skilled in the art that there is no absolute distinction between “highly stringent conditions” and “moderately stringent conditions.” For example, at 0.015 M sodium ion (no formamide), the melting temperature of perfectly matched long DNA is about 71° C. With a wash at 65° C. (at the same ionic strength), this would allow for approximately a 6% mismatch. To capture more distantly related sequences, one skilled in the art can simply lower the temperature or raise the ionic strength.

A good estimate of the melting: temperature in 1M NaCl* for oligonucleotide probes up to about 20 nt is given by: Tₘ=2° C. per A-T base pair+4° C., per G-C base pair *The sodium ion concentration in 6.times. salt sodium citrate (SSC) is 1M, See Suggs et al., Developmental Biology Using Purified Genes 683 (Brown and Fox, eds., 1981).

High stringency washing conditions for oligonucleotides may be at a temperature of 0-5° C. below the Tm of the oligonucleotide, e.g., in 6×SSC, 0.1% SDS.

### Exemplary Methods

Efficient molecular analysis usually requires detecting the presence of an analyte in a very small sample at very low concentration. The invention provides a method for detecting the presence of a polymeric analyte, e.g., nucleic acid, e.g., DNA or RNA, lipid, for instance a fatty acid, polysaccharide, protein, or peptide, in a sample. The method includes contacting the sample with magnetic particles, such as beads, in a detection chamber, subjecting the sample to energy, e.g., a rotating magnetic field, acoustic energy or vibration, so as to induce aggregation, for instance by placing the detection chamber at approximately the center of a rotating magnetic field, and detecting the presence of aggregates, e.g., pinwheels, which indicates the presence of the polymeric analyte. The use of an external magnetic field in microdevices to implement magnetic bead control has previously been disclosed, e.g., by U.S. Pat. Nos. 7,452,726; 6,664,104; 6,632,655; and 6,344,326; which are incorporated herein by reference. In one embodiment, the energy is emitted is a magnetic field, such as one generated by a U-shaped magnet. In one embodiment, the magnetic beads are coated. or derivatized to bind or enhance binding to the polymeric analyte. In one embodiment, the magnetic beads are coated with silica, amine-based charge switch, boronic acid, silane, oligonucleotides, lectins, reverse phase, antibody, antigen, avidin, or biotin. In one embodiment, the detection chamber is part of a microfluidic device. In one embodiment, the polymeric analyte is nucleic acid, the magnetic beads are silica coated, and the contacting occurs in the presence of a chaotropic agent. In one embodiment, the polymeric analyte is positively charged polysaccharide, the magnetic beads are silica coated, and the contacting occurs under low ionic strength conditions. In one embodiment, the polymeric analyte is protein, the magnetic beads are silica coated, and the contacting occurs under denaturing conditions for the protein.

In one embodiment, the present invention uses magnetic beads in a rotating magnetic field to provide a visual detection of the presence or quantity of a polymeric analyte, such as nucleic acids, lipids, polysaccharides, proteins, etc, although any source of energy that induces aggregation, such as acoustic energy or vibration may be employed. This method arises from the observation that when a polymeric analyte binds to the magnetic beads, application of a rotating magnetic field to the beads results in unique pinwheel-like formations. Without the presence of the polymeric analyte, the movement and conformation of the magnetic beads induced by the rotating magnetic field (non-aggregated) differs significantly from the pinwheel formations. As such, the pinwheel formation is specific to the presence of the binding between the polymeric analyte and the magnetic beads, and the rotating magnetic field, and therefore, can be used to detect the presence of the analyte. However, aggregate formation is not specific for a rotating magnetic field, and may be induced by other means, e.g., by an external acoustic force or vibration. Pinwheel formation in a mixture with a polymeric analyte may be enhanced by applying other forms of energy, e.g., by vibrating the sample.

In one embodiment, the present invention relates to a method for detecting the presence of polymeric analyte in a complex biological sample by contacting the sample with magnetic beads, or another magnetic solid substrate that can be suspended in solution, and exposing the magnetic beads to a rotating magnetic field. The presence of pinwheel formations indicates the presence of the bound polymeric analyte. In one embodiment, the magnetic beads are coated or derivatized to specifically bind or to enhance the binding of the polymeric analyte to the magnetic beads. The environment can also be manipulated to enhance the binding of the polymeric analyte to the magnetic beads.

The present invention also relates to a system for detecting the presence of a polymeric analyte in a complex liquid biological sample. The system contains a rotatable magnet, e.g., one mounted on a motor, so that, when activated, the motor rotates the magnet to create a rotating magnetic field. The system also contains a detection chamber, containing magnetic beads therein, located approximately at the center of the magnet, between its north and south poles. In use, sample is placed into the detection chamber. The motor is then activated to rotate the magnet around the detection chamber. The presence of pinwheel formations in the chamber indicates the presence of the polymeric analyte in the sample.

The method and apparatus of the invention can be added onto already existing assays or apparatuses, especially a micro-total analysis system (μ-TAS), to act as a polymeric analyte detector. For example, the presence of an antibody/antigen reaction may initiate the coupling of nucleic acids and the presence/absence of the pinwheel formations determines whether the antibody/antigen binding has occurred. This is analogous to an immuno-PCR method, where instead of using PCR and fluorescent probes for the detection of nucleic acids, the pinwheel formations are employed.

The present invention is based on the observation that polymeric analytes, when bound to magnetic beads and in the presence a rotating magnetic field, produce unique pinwheel formations. The pinwheel effect is not seen in a static magnetic field and appears to be specific to a rotating magnetic field. “Pinwheel formation” as used herein refers to a rotating mass having a circular or disc-like cross-section. The mass is made of clumps or aggregates of magnetic beads tethered by a polymeric analyte. When viewed, in a still photograph, the pinwheel formation looks like a disc shaped object made of an aggregate of magnetic beads. However, when viewed visually or by imaging, the disc shaped object actually spins around its center axis similar to that of a spinning pinwheel. Within a detection chamber, the pinwheel formations sometimes collide together to form larger pinwheels, and sometimes collide with the wall of the chamber to break up into smaller pinwheels.

An apparatus for practicing the methods of the present invention includes a rotatable magnet, preferably mounted on a motor, and a detection chamber located approximately at the center of the magnet, between its north and south pole. In one embodiment, the apparatus contains a stir plate, having a rotatable magnet therein, and a detection chamber placed at the center of the stir plate. The stir plate has a top cover, on top of which the detection chamber sits. In one embodiment, underneath to top cover sits a magnet having a north pole and a south pole. The magnet may be a U-shaped magnet having its poles at either end of the U, however other magnet shapes may be used, e.g., I-shape or semicircular shape magnets. The magnet may be a motor that is capable of rotating the magnet around its center axis. The magnet may be located directly below the detection chamber, nevertheless other configurations may be used as long as the detection chamber is located approximately between the two poles of the magnet. The magnetic field may be positioned either parallel, orthogonal or at any angle to the detection chamber. The beads move in a defined form, where they form a pinwheel structure and spin in a distinct direction correlating to the directional rotating of the magnetic field. A rotatable magnet or other devices that can produce a rotating magnetic field may be employed. Such devices may be an electromagnet or electronic circuitry that can produce a rotating magnetic field similar to that produced by the rotating magnet or electromagnetic induction.

The detection chamber may be any fluid container that can be placed at approximately the center of the magnet (approximately the center of the magnetic field when the magnet is rotating). The detection chamber may be part of or a component of a microfluidic device or micro-total analysis system (μ-TAS). Generally, a microfluidic device or μ-TAS contains at least one micro-channel. There are many formats, materials, and size scales for constructing μ-TAS. Common μ-TAS devices are disclosed in U.S. Pat. No. 6,692,700 to Handique et al.; U.S. Pat. No. 6,919,046 to O'Connor et al; U.S. Pat. No. 6,551,841 to Wilding et al.; U.S. Pat. No. 6,630,353 to Parce et al.; U.S. Pat. No. 6,620,625 to Wolk et al.; and U.S. Pat. No. 6,517,234 to Kopf-Sill et al.; the disclosures of which are incorporated herein by reference. Typically, a μ-TAS device is made up of two or more substrates that are bonded together. Microscale components for processing fluids are disposed on a surface of one or more of the substrates. These microscale components include, but are not limited to, reaction chambers, electrophoresis modules, microchannels, reservoirs, detectors, valves, or mixers. When the substrates are bonded together, the microscale components are enclosed and sandwiched between the substrates. A detection chamber may include a microchannel. At both ends of the microchannel are inlet and outlet ports for adding and removing samples from the microchannel. The detection chamber may be linked to other microscale components of a μ-TAS as part of an integrated system for analysis.

The detection chamber may contain magnetic beads prior to the addition of the sample or the magnetic beads may be added to the detection chamber along with the sample. The magnetic beads may contain a surface that is derivatized or coated with a substance that binds or enhances the binding of the polymeric analyte to the magnetic beads. Some coatings or derivatizations include, but are not limited to, amine-based charge switch, boronic acid, silanization, reverse phase, oligonucleotide, lectin, antibody-antigen, peptide-nucleic acid (PNA)-oligonucleotide, locked nucleic acid (LNA)-oligonucleotide, and avidin-biotin. For example, for the detection of nucleic acid, the magnetic beads can be silica coated to specifically bind nucleic acids when exposed to a high ionic strength, chaotropic buffer. A bead may also be coated with positively charged amines or oligomers for binding with nucleic acids.

To bind carbohydrates, the magnetic beads may contain a boronic acid-modified surface. Boronic acid bonds covalently and specifically to -cis dialcohols, a moiety common in certain carbohydrates including glucose.

To bind lipids, the magnetic beads may be modified with hydrophobic groups, such as benzyl groups, alkanes of various lengths (6-20), or vinyl groups. The lipids are bound to the beads by hydrophobic forces.

To bind proteins, the magnetic beads may contain a protein modified surface. For example, the surface of the beads may be coated with an antibody specific for the protein of interest. For general protein detection, the bead surface may be coated with avidin or biotin and the protein of interest may be derivatized with biotin or avidin. The avidin-biotin binding thus allows the protein to bind to the beads.

In addition to derivatization or coating of the magnetic beads, the physical environment where the polymeric analyte comes into contact with the magnetic beads may also be altered to allow the beads to specifically bind or to enhance the binding of the magnetic beads to the polymeric analyte. For example, a silica coated bead may be manipulated to specifically hind nucleic acid, carbohydrate, or protein depending on the conditions used: binding of DNA occurs in chaotropic salt solution, binding of positively charged carbohydrates occurs in low ionic strength solutions, and binding of proteins occurs under denaturing conditions (in the presence of urea, heat, and the like).

Depending on the concentration of polymeric analyte to be detected, the number of beads in the channel may be about 100 to about 10⁸, such as about 10⁴ to 10⁷ for visual detection. Fluorescence detection may allow for a smaller number of beads, e.g., about 10. The higher the concentration of analyte in the sample, the higher the amount of magnetic beads that should be employed.

The components of the magnetic field in the x-axis and z-axis are essentially negligible in the center of the magnetic field and thus are likely not critical to pinwheel formation. The magnetic field in the y-axis may have a strength of about 1 to 5,000 gauss, more preferably about 10 to 1000 gauss. Additionally, regardless of the shape of the magnet, the magnetic field component in the y-axis may obtain its maximum strength at the center of rotation and is at its minimum strength at both poles of the magnet. The field component may be maximized along the length of the magnet and may abruptly drop to its minimum at the poles. The field component does not significantly decrease off either side of the magnet. The magnetic field lines at the detection chamber may be parallel to the xy-plane in which the detection chamber lies.

To detect the polymeric analyte in a sample, the sample is added to the detection chamber, The detection chamber may already contain magnetic beads therein or the magnetic beads may be added to the chamber along with the sample. With the chamber locating at approximately the center of the magnet (between the two poles of the magnet), the magnet is rotated so that the chamber experiences a rotating magnetic field (the rotating magnetic field can also be effected using electronic circuitry rather than a magnet). The magnet may be rotated at about 10 to 10,000 rpm, such as at about 1000 to 3000 rpm. Observation of pinwheel formations in the channel indicates the presence of the polymeric analyte in the sample. The average size (diameter) of the pinwheels may be proportional to the concentration of polymeric analyte, e.g., nucleic acids, in the sample. A calibration curve may be obtained for correlating the average size of the pinwheels to the polymeric analyte concentration. Such a calibration curve may be generated, for example, by subjecting known concentrations of the polymeric analyte to the rotating magnetic field and determining the average size of the pinwheel formations for each concentration.

The presence of pinwheel formations can be detected visually, or using optical or imaging instrumentation. One way to detect pinwheel formations is to photograph or record a video of the detection chamber. This may be accomplished by the image or recording of one chamber at a time or multiple chambers. A computer program can then be used to detect the pinwheel formations in the photograph or video. The program may initially upload a d crop the image (photograph or frames of a video) so that only the detection chamber is shown. The cropped image may then converted to gray scale. An extended minima transformation is then performed with a threshold between about 40 to 70 to isolate the magnetic microparticles from the background pixels. Once holes within each object are filled in, each object may then be labeled, e.g., with a separate RGB color. A boundary is then created around each distinct object. For each boundary, a metric m=4πa/p² is calculated, where a is the area of the object and p is the perimeter of the object. The metric m is a measure of the roundness of the object, for a perfect circle m=1. For each object, if m is greater than about 0.8, such as greater than about 0.95, that object is defined as a pinwheel. A centroid is then plotted over each object having m greater than about 0.8 (a pinwheel). If a photograph is used, the number of pinwheels is then counted. If a video is used, the steps are repeated for each frame of the video and the average number of pinwheels per frame is calculated. If the number of pinwheels or average number of pinwheels per frame is greater than a set value from 0.5 to 10 (depending upon the polymeric analyte and bead concentration), the program returns the result that polymeric analyte is present in the sample. See, for example, WO 2009/114709, the disclosure of which is incorporated by reference herein.

For software based automated detection, one possible system contains at least a camera and a computer for running the computer program. In this system, the camera takes pictures or video of the detection chamber and the images from the camera is analyzed by the computer. The computer is preferably electronically connected to the camera for automatically downloading and processing the images from the camera as discussed above. The automated detection is especially efficient when the detection chamber is part of a μ-TAS where the computer can also be use to control and sense other aspects of the μ-TAS, such as temperature, fluid flow, gating, reaction monitoring, etc.

### Particles

Particles useful in the practice of the invention include metal (e.g., gold, silver, copper and platinum), semiconductor (e.g., CdSe, CdS, and CdS or CdSe coated with ZnS) and magnetic (e.g., ferromagnetite) as colloidal materials, as well ZnS, ZnO, TiO2, AgI, AgBr, HgI2, PbS, PbSe, ZnTe, CdTe, In2S3, In2 Se3, Cd3 P2, Cd3 As2, InAs, and GaAs, and silica and polymer (e.g., latex) particles. The particles may have any shape, e.g., spheres (generally referred to as beads) or rods, or irregular shapes, and a population of particles may have particles that vary in shape or size, e.g., beads in a population of beads may not have a uniform shape or diameter. The size of the particles may be from about 1 nm to about 300 micrometers (μm) (mean diameter for rods or spheres), such as from about 0.5 to about 250 μm, or from about 2 to about 10 μm. The particles may be coated or derivatized with agents, e.g., to enhance binding of a selected analyte. For example, particles may include a silica coating or be derivatized with streptavidin.

In various aspects, the methods provided include those utilizing particles which range in size from about 1 micrometers to about 250 micrometers in mean diameter, about 1 micrometers to about 240 micrometers in mean diameter, about 1 micrometers to about 230 micrometers in mean diameter, about 1 micrometers to about 220 micrometers in mean diameter, about 1 micrometers to about 210 micrometers in mean diameter, about 1 micrometers to about 200 micrometers in mean diameter, about 1 micrometers to about 190 micrometers in mean diameter, about 1 micrometers to about 180 micrometers in mean diameter, about 1 micrometers to about 170 micrometers in mean diameter, about 1 micrometers to about 160 micrometers in mean diameter, about 1 micrometers to about 150 micrometers in mean diameter, about 1 micrometers to about 140 micrometers in mean diameter, about 1 micrometers to about 130 micrometers in mean diameter, about 1 micrometers to about 120 micrometers in mean diameter, about 1 micrometers to about 110 micrometers in mean diameter, about 1 micrometers to about 100 micrometers in mean diameter, about 1 micrometers to about 90 micrometers in mean diameter, about 1 micrometers to about 80 micrometers in mean diameter, about 1 micrometers to about 70 micrometers in mean diameter, about 1 micrometers to about 60 micrometers in mean diameter, about 1 micrometers to about 50 micrometers in mean diameter, about 1 micrometers to about 40 micrometers in mean diameter, about 1 micrometers to about 30 micrometers in mean diameter, or about 1 micrometers to about 20 micrometers in mean diameter, about 1 micrometers to about 10 micrometers in mean diameter. In other aspects, the size of the particles is from about 5 micrometers to about 150 micrometers, from about 5 to about 50 micrometers, from about 10 to about 30 micrometers. The size of the particles is from about 5 micrometers to about 150 micrometers, from about 30 to about 100 micrometers, from about 40 to about 80 micrometers. In one embodiment, the magnetic particle may have an effective diameter of about 0.25 to 50 micrometers, including from about 0.5 to about 1.5 micrometers or from about 3 to about 15 micrometers, The size of the beads may be matched with the expected size of the polymeric analyte, e.g., nucleic acid, being detected. Smaller beads form pinwheels with shorter polymer analytes and smaller beads may be more sensitive to shorter polymeric analytes. Bead size can be tuned. to the specific cutoff in size needed for discrimination, including optical properties or amount surface area that can be derivatized.

In one embodiment, MagneSil particles (Promega Corp, Madison, Wis.) are employed. MagneSil particles are paramagnetic particles (iron-cored silicon dioxide beads) of about 8 micrometers in average diameter with the overall range of about 4 to about 12 microns in diameter. Those particles can be loaded into a microchip chamber and contacted with sample DNA, and then subjected to a magnetic field from an external magnet.

### Oligonucleotides

Methods of making oligonucleotides of a predetermined sequence are well-known. See, for example, Sambrook et al., Molecular Cloning: A Laboratory Manual (2nd ed. 1989) and Eckstein (ed.) Oligonucleotides and Analogues, 1st Ed. (Oxford University Press, New York, 1991). Solid-phase synthesis methods are contemplated for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically. Non-naturally occurring nucleobases can be incorporated into the oligonucleotide, as well. See, e.g., Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

The term “oligonucleotide” as used herein includes modified forms as discussed herein as well as those otherwise known in the art which are used to regulate gene expression. Likewise, the term “nucleotides” as used herein is interchangeable with modified forms as discussed herein and otherwise known in the art. In certain instances, the art uses the term “nucleobase” which embraces naturally-occurring nucleotides as well as modifications of nucleotides that can be polymerized. Herein, the terms “nucleotides” and “nucleobases” are used interchangeably to embrace the same scope unless otherwise noted.

In various aspects, the methods may employ oligonucleotides which are DNA oligonucleotides, RNA oligonucleotides, or combinations of the two types. Modified forms of oligonucleotides are also contemplated which include those having at least one modified internucleotide linkage. In one embodiment, the oligonucleotide is all or in part a peptide nucleic acid (PNA) or includes LNA (see Koskin et al., Tetrahedron, 54:3607 (1998)). Other modified internucleoside linkages include at least one phosphorothioate linkage. Still other modified oligonucleotides include those comprising one or more universal bases, “Universal base” refers to molecules capable of substituting for binding to any one of A, C, G, T and U in nucleic acids by forming hydrogen bonds without significant structure destabilization. The oligonucleotide incorporated, with the universal base analogues is able to function as a probe in hybridization, as a primer in PCR and DNA sequencing. Examples of universal bases include but are not limited to 5′-nitroindole-2′-deoxyriboside, 3-nitropyrrole, inosine, and hypoxanthine.

Modified Backbones. Specific examples of oligortucleotides include those containing modified backbones or non-natural internucleoside linkages. Oligonucleotides having modified backbones include those that retain a phosphorus atom in the backbone and those that do not have phosphorus atom in the backbone. Modified oligonucleotides that do not have a phosphorus atom in their internucleoside backbone are considered to be within the meaning of “oligonucleotide.”

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

Modified Sugar and Internucleoside Linkages. In still other embodiments, oligonucleotide mimetics wherein both one or more sugar and/or one or more internucleotide linkage of the nucleotide units are replaced with “non-naturally occurring” groups. In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still other embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligo consists of 2 to 4, desirably 3, groups/atoms selected from —CH₂—, —O—, —S—, —NR^{H}—C═O, C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂ CH=(including R⁵ when used as a linkage to a succeeding monomer), —CH₂—CH₂O—, —NR^{H}—CH₂—CH₂—, —CH₂—CH₂—NR^{H}—, —CH₂—NR^{H}—CH₂—, —O—CH₂—CH₂—NR^{H}—CO—O—, —NR^{H}—CO—NR^{H}—, —NR^{H—CS—NR}^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H—CO—CH}₂—, —O—CH₂—Co—NR^{H—, —O—CH}₂—CH₂—NR^{H}13 , —CH═N—O—, —Ch₂—NR^{H}—O—, —CH₂—O—N=(including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH=(including R⁵ when used as a linkage to a succeeding monomer), —S—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}13 S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(CH₂CH₃)—O—, —O—PO(OCH₂CH₂S—R)—O—, —O—PO(BH₃)—O—, —O—PO(NHR^{N})—O—, —O—P(O)₂—NR^{H}H—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H}P(O)₂—O—, —O—P(O,NR^{H})—O—, —O—PO(R″)—O—, —O—PO(CH₃)—O—, and —O—PO(NHR^{N})—O—, where RH is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology, 5:343-355 (1995) and Susan M. Freier and Karl-Heinz Altmann, Nucleic Acids Research, 25:4429-4443 (1997).

Still other modified forms of oligonucleotides are described in detail in U.S. Patent Publication No. 20040219565, the disclosure of which is incorporated by reference herein in its entirety.

Modified oligonucleotides may also contain one or more substituted sugar moieties. in certain aspects, oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl, and alkynyl. Other embodiments include O[(CH₂)ₙO]ₘCH₃, O(CH₂)ₙOCH₃, O(CH₂)ₙNH₂, O((CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one aspect, a modification includes 2-methoxyethoxy (2′-O—CH₂CH₂OCH₃, also known as 2′-O-(2-methoxyethyl) or 2′-MOE) (Martin et al., Helv. Chim. Acta, 78:486-504 (1995)) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples herein below.

Still other modifications include T-methoxy (2′-O—CH₃), 2′-aminopropoxy (2-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Natural and Modified Bases. Oligonucleotides may also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyritnidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone, Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30:613 (1991), and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1,2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

A “modified base” or other similar term refers to a composition which can pair with a natural base (e.g., adenine, guanine, cytosine, uracil, and/or thymine) and/or can pair with a non-naturally occurring base. In certain aspects, the modified base provides a Tₘ differential of 15, 12, 10, 8, 6, 4, or 2° C. or less. Exemplary modified bases are described in EP 1 072 679 and WO 97/12896.

An oligonucleotide, or modified form thereof, may be from about 20 to about 100 nucleotides in length. In one embodiment, the oligonucelotide is from 5 to 50 nucleotides in length or any integer in between. It is also contemplated wherein the oligonucleotide is about 20 to about 90 nucleotides in length, about 20 to about 80 nucleotides in length, about 20 to about 70 nucleotides in length, about 20 to about 60 nucleotides in length, about 20 to about 50 nucleotides in length about 20 to about 45 nucleotides in length, about 20 to about 40 nucleotides in length, about 20 to about 35 nucleotides in length, about 20 to about 30 nucleotides in length, about 20 to about 25 nucleotides in length, or about 15 to about 90 nucleotides in length, about 15 to about 80 nucleotides in length, about 15 to about 70 nucleotides in length, about 15 to about 60 nucleotides in length, about 15 to about 50 nucleotides in length about 15 to about 45 nucleotides in length, about 15 to about 40 nucleotides in length, about 15 to about 35 nucleotides in length, about 15 to about 30 nucleotides in length, about 15 to about 25 nucleotides in length, or about 15 to about 20 nucleotides in length, and all oligonucleotides intermediate in length of the sizes specifically disclosed to the extent that the oligonucleotide is able to achieve the desired result. Accordingly, oligonucleotides of 15, 16, 17, 18, 19, 20, 21. 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleotides in length are contemplated.

“Hybridization,” which is used interchangeably with the term “complex formation” herein, means an interaction between two or three strands of nucleic acids by hydrogen bonds in accordance with the rules of Watson-Crick DNA complementarity, Hoogstein binding, or other sequence-specific binding known in the art. Hybridization can be performed under different stringency conditions known in the art.

In various aspects, the methods include use of oligonucleotides which are 100% complementary to another sequence, i.e., a perfect match, while in other aspects, the individual oligonucleotides are at least (meaning greater than or equal to) about 95% complementary to all or part of another sequence, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to that sequence, so long as the oligonucleotide is capable of hybridizing to the target sequence.

It is understood in the art that the sequence of the oligonucleotide used in the methods need not be 100% complementary to a target sequence to be specifically hybridizable. Moreover, an oligonucleotide may hybridize to a target sequence over one or more segments such that intervening or adjacent segments are not involved in the hybridization event (e.g., a loop structure or hairpin structure). Percent complementarity between any given oligonucleotide and a target sequence can be determined routinely using BLAST programs (basic local alignment search tools) and PowerBLAST programs known in the art (Altschul et at, J. Mol. Biol., 215: 403-410 (1990); Zhang and Madden, Genome Res., 7:649-656 (1997)).

The stability of the hybrids is chosen to be compatible with the assay conditions. This may be accomplished by designing the nucleotide sequences in such a way that the Tₘ will be appropriate for standard conditions to be employed in the assay. The position at which the mismatch occurs may be chosen to minimize the instability of hybrids. This may be accomplished by increasing the length of perfect complementarity on either side of the mismatch, as the longest stretch of perfectly homologous base sequence is ordinarily the primary determinant of hybrid stability. In one embodiment, the regions of complementarity may include G:C rich regions of homology. The length of the sequence may be a factor when selecting oligonucleotides for use with particles. In one embodiment, at least one of the oligonucleotides has 100 or fewer nucleotides, e.g., has 15 to 50, 20 to 40, 15 to 30, or any integer from 15 to 50, nucleotides. Oligonucleotides having extensive self-complementarity should be avoided. Less than 15 nucleotides may result in a oligonucleotide complex having a too low a melting temperature to be suitable in the disclosed methods. More than 100 nucleotides may result in a oligonucleotide complex having a too high melting temperature to be suitable in the disclosed methods. Thus, oligonucleotides are of about 15 to about 100 nucleotides, e.g., about 20 to about 70, about 22 to about 60, or about 25 to about 50 nucleotides in length.

### Particles for Hybridization Induced Aggregation

A functionalized particle has at least a portion of its surface modified, e.g., with an oligonucleotide. In one embodiment, any particle having oligonucleotides attached thereto suitable for use in detection assays and that do not interfere with oligonucleotide complex formation, i.e., hybridization to form a double-strand complex.

For a hybridization induced aggregation assay, at least two types of particles having attached thereto oligonucleotides with sequences (a and b) complementary to a target nucleic acid sequence (having a′ and b′) are prepared. In one embodiment, the oligonucleotides a and b are functionalized to two types of particles in away that oligonucleotide a is attached to the particle by its 3′ OH group, and oligonucleotide b is attached to the particle by the 5′ PO₄³-group.

In various aspects, at least one oligonucleotide is bound through a spacer to the particle. In these aspects, the spacer is an organic moiety, a polymer, a water-soluble polymer, a nucleic acid, a polypeptide, and/or an oligosaccharide. Methods of functionalizing the oligonucleotides to attach to a surface of a particle are well known in the art. See Whitesides, Proceedings of the Robert A. Welch Foundation 39th Conference On Chemical Research Nanophase Chemistry, Houston, Tex., pages 109-121 (1995). See also, Mucic et al., Chem. Comm. 555-557 (1996) (describes a method of attaching 3′ thiol DNA to flat gold surfaces; this method can be used to attach oligonucleotides to particles). The alkanethiol method can also be used to attach oligonucleotides to other metal, semiconductor and magnetic colloids and to the other particles listed above. Other functional groups for attaching oligonucleotides to solid surfaces include phosphorothioate groups (see, e.g., U.S. Pat. No. 5,472,881 for the binding of oligonucleotide-phosphorothioates to gold surfaces), substituted alkylsiloxanes (see, e.g. Burwell, Chemical Technology, 4:370-377 (1974) and Matteucci and Caruthers, J. Am. Chem. Soc., 103:3185-3191 (1981) for binding of oligonucleotides to silica and glass surfaces, and Grabaretal, Anal. Chem., 67:735-743 for binding of aminoalkylsiloxanes and for similar binding of mercaptoaklylsiloxanes). Oligonucleotides terminated with a 5′ thionucleoside or a 3′ thionucleoside may also be used for attaching oligonucleotides to solid. surfaces. The following references describe other methods which may he employed to attach oligonucleotides to particles: Nuzzo et al., J. Am. Chem. Soc., 109:2358 (1987) (disulfides on gold); Allara and Nuzzo, Langmuir, 1:45 (1985) (carboxylic acids on aluminum); Allara and Tompkins, J. Colloid Interface Sci., 49; 410-421 (1974) (carboxylic acids on copper); Iler, The Chemistry Of Silica, Chapter 6, (Wiley 1979) (carboxylic acids on silica); Timmons and Zisman, J. Phys. Chem., 69:984-990 (1965) (carboxylic acids on platinum); Soriaga and Hubbard, J. Am. Chem. Soc., 104:3937 (1982) (aromatic ring compounds on platinum); Hubbard, Acc. Chem. Res., 13:177 (1980) (sulfolanes, sulfoxides and other functionalized solvents on platinum); Hickman et al., J. Am. Chem. Soc., 111:7271 (1989) (isonitriles on platinum); Maoz and Sagiv, Langmuir, 3:1045 (1987) (silanes on silica); Maoz and Sagiv, Langmuir, 3:1034 (1987) (silanes on silica); Wasserman et al., Langmuir, 5:1074 (1989) (silanes on silica); Eltekova and Eltekov, Langmuir, 3:951 (1987) (aromatic carboxylic acids, aldehydes, alcohols and methoxy groups on titanium dioxide and silica); Lee et al., J. Phys. Chem., 92:2597 (1988) (rigid phosphates on metals).

The particles, the oligonucleotides or both are functionalized in order to attach the oligonucleotides to the particles. Such methods are known in the art. Each particle will have a plurality of oligonucleotides attached to it. As a result, each particle-oligonucleotide conjugate can bind to a plurality of oligonucleotides or nucleic acids having the complementary sequence.

The following examples are given to illustrate the present invention. It should be understood that the invention is not to be limited to the specific conditions or details described in those examples.

### Example I

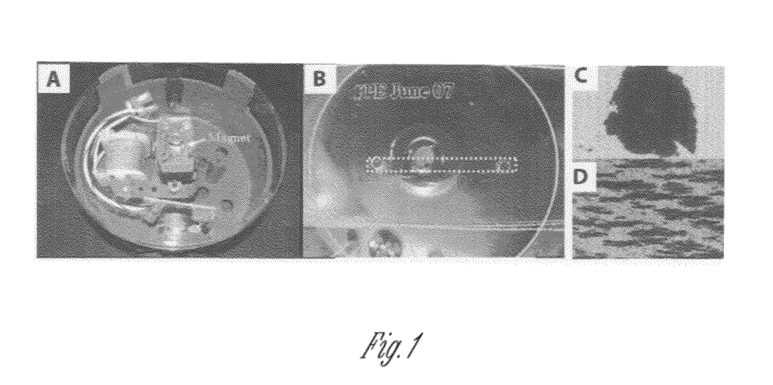

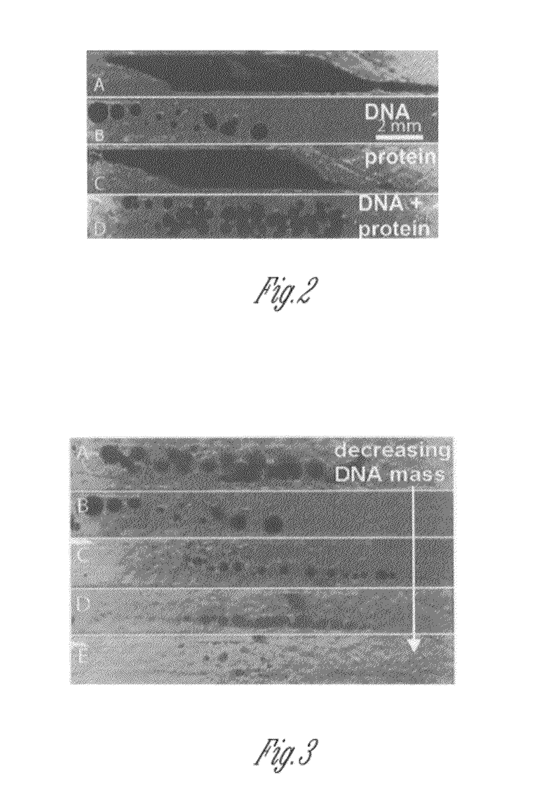

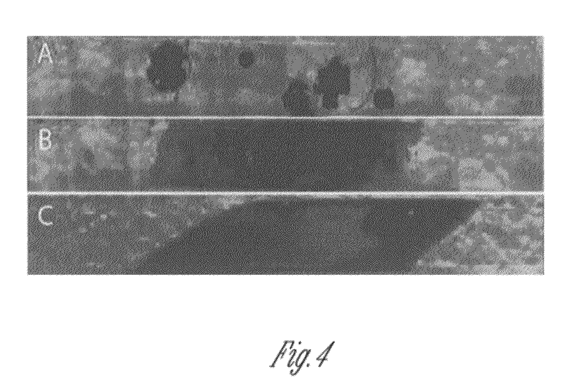

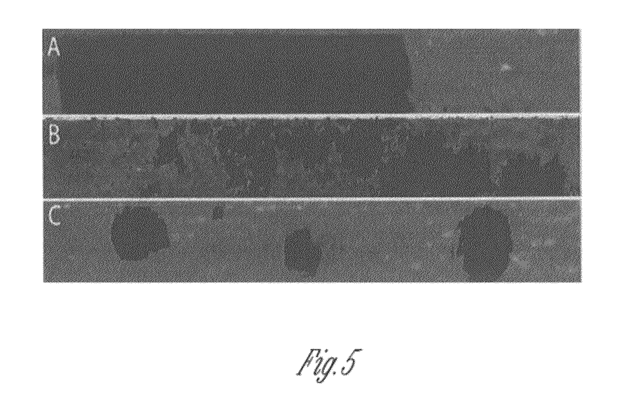

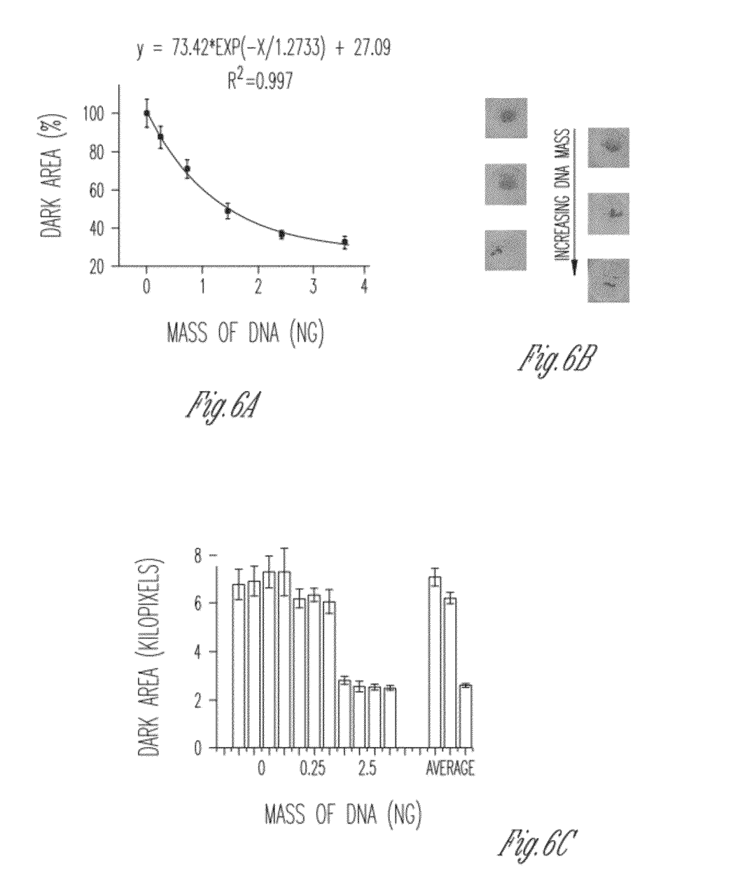

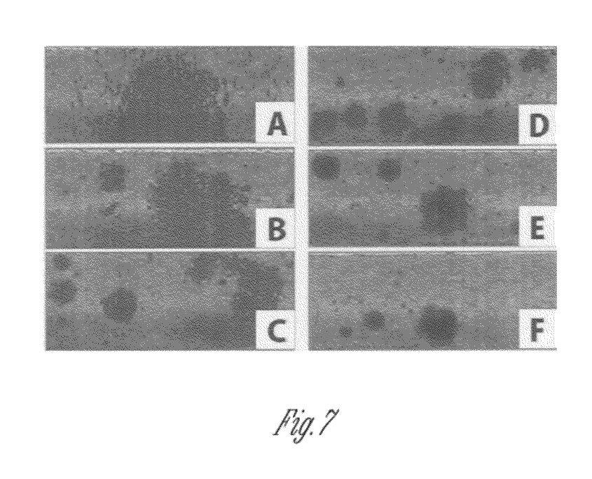

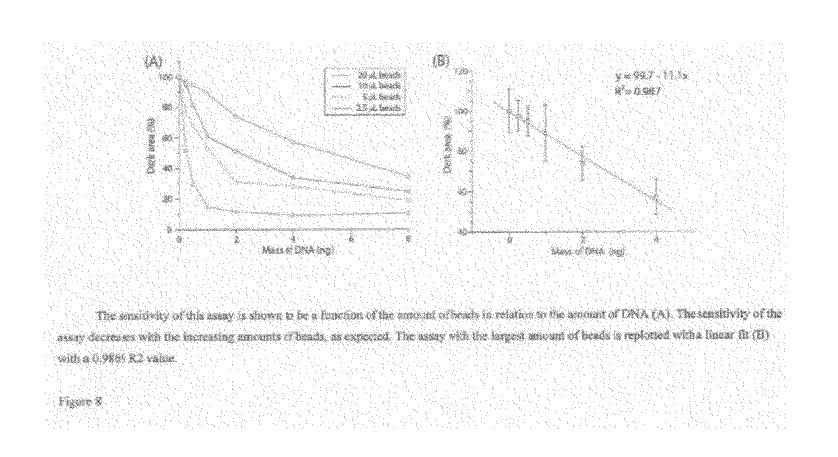

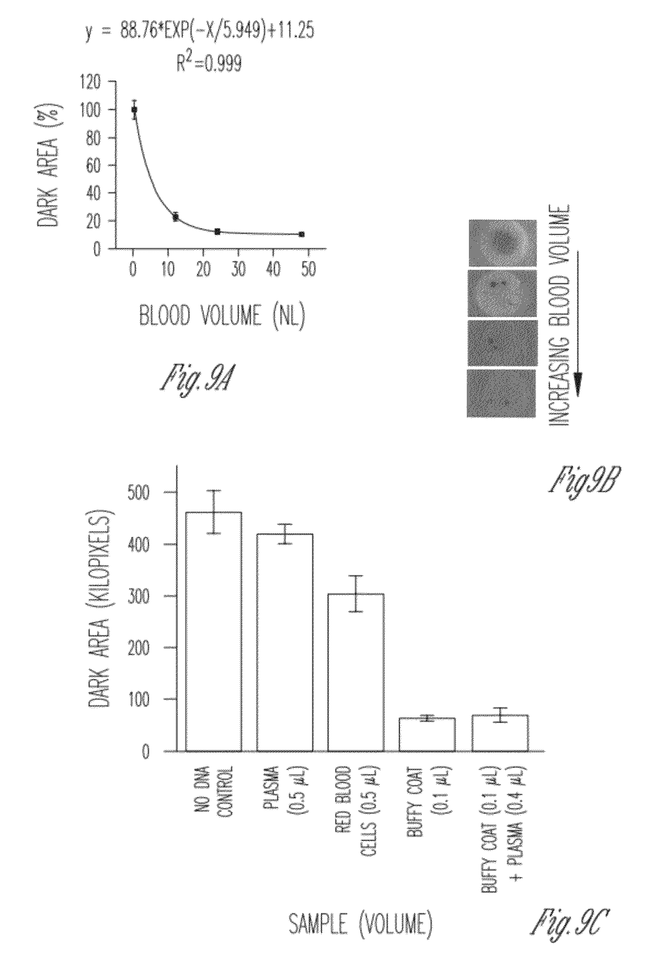

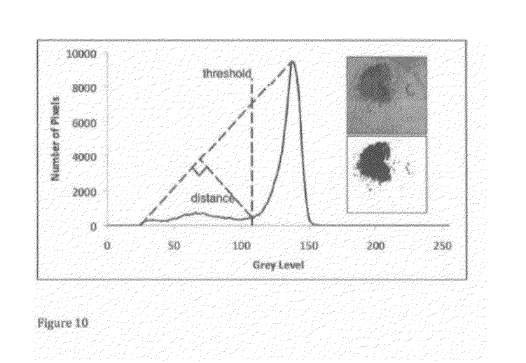

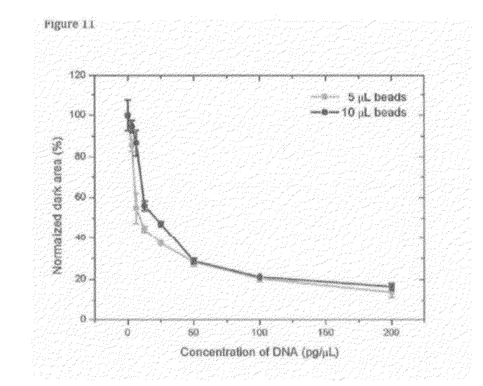

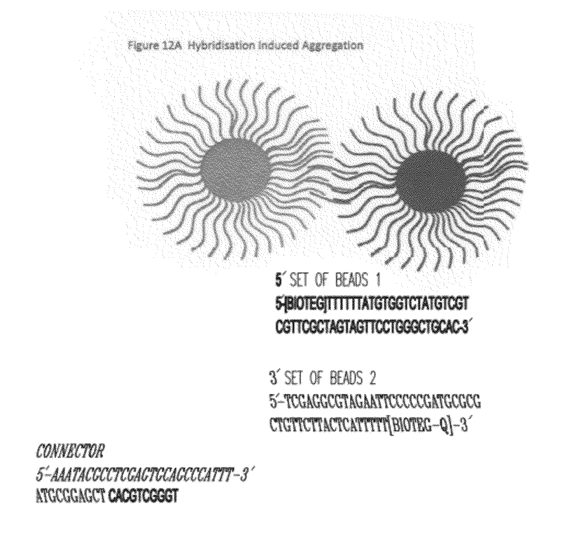

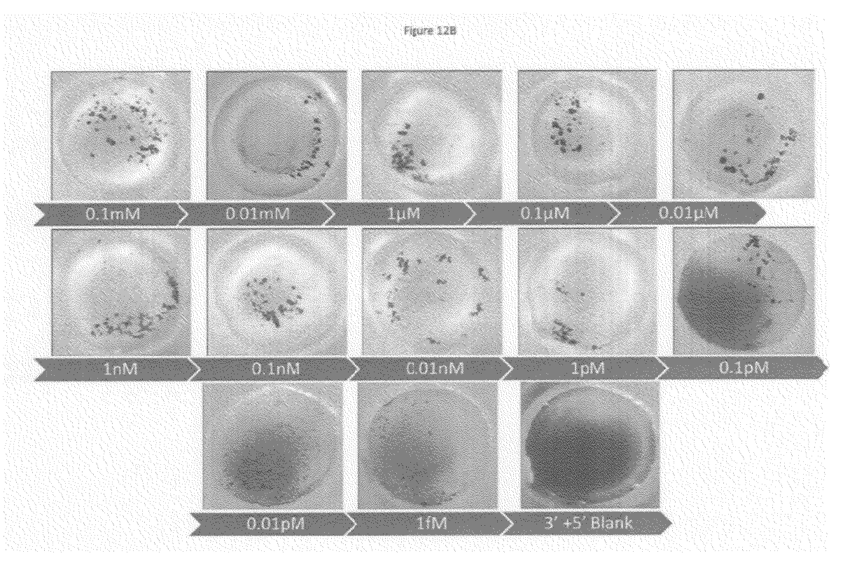

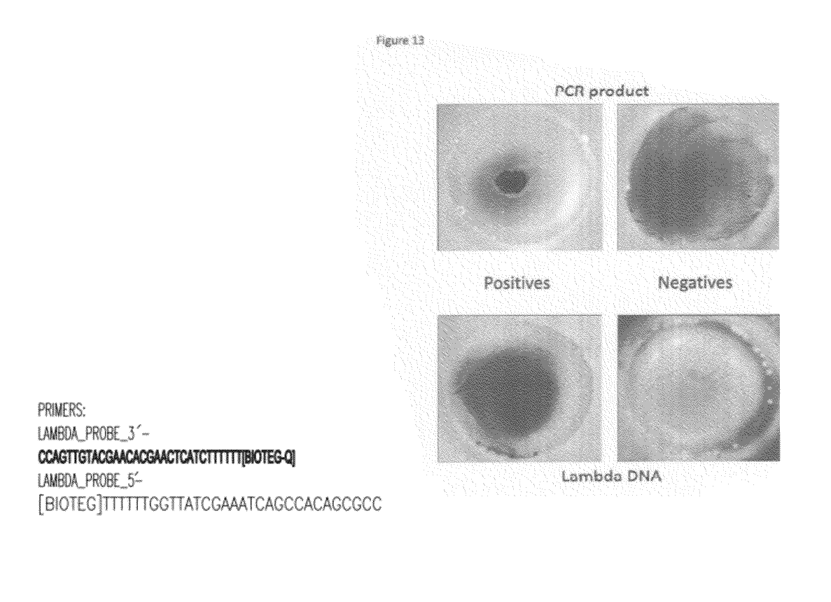

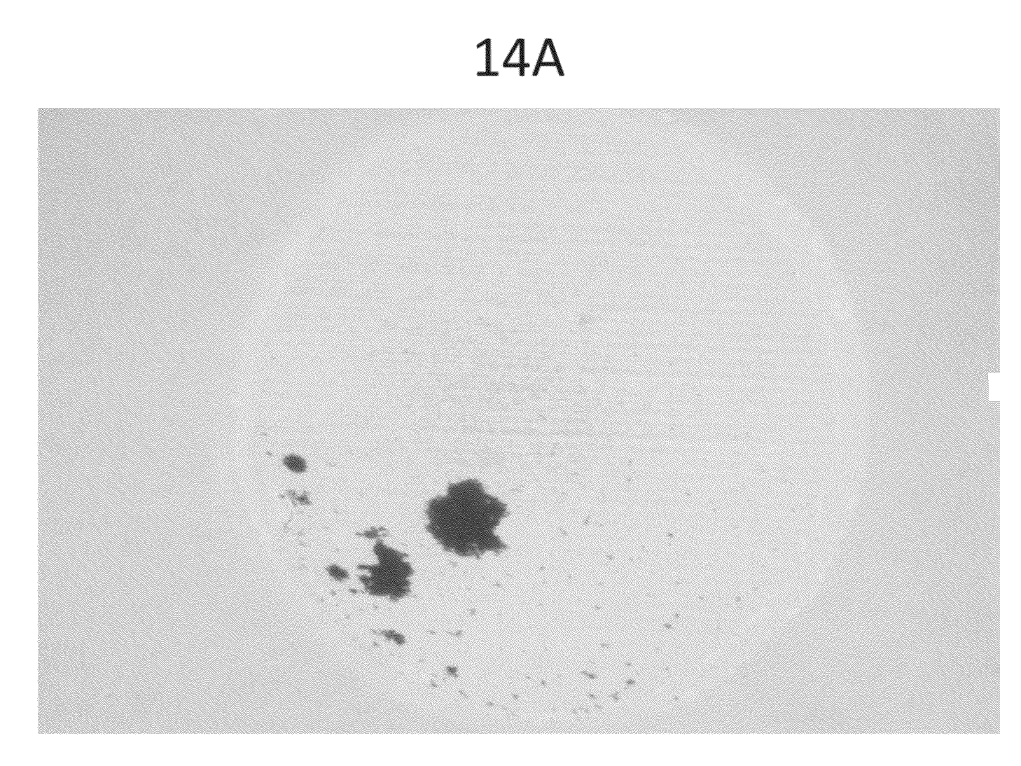

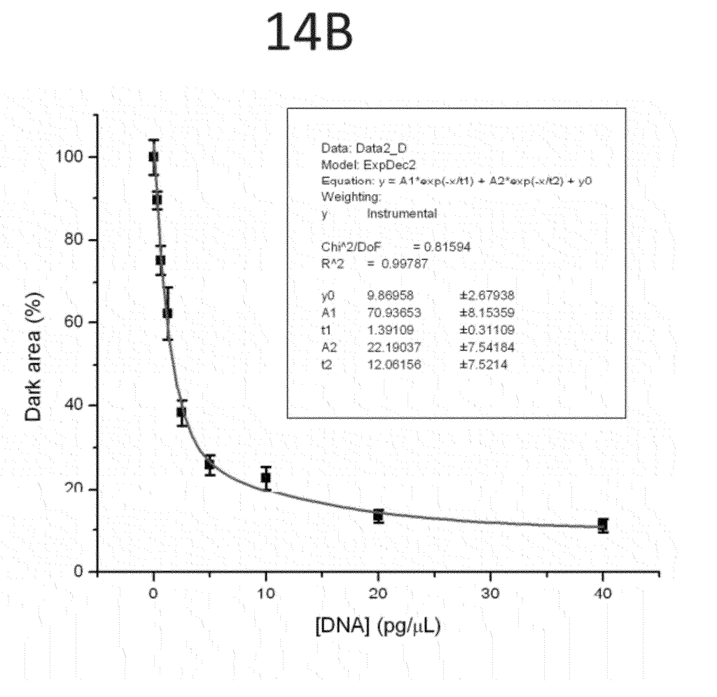

A RMF centered on a microfluidic chamber containing a minute mass of magnetic silica beads (FIG. 1) reveals the presence of a select polymeric analyte in the sample through bead aggregation and the formation of ‘pinwheels’ (FIG. 2B). When the sample is devoid of specific polymeric analytes, the beads remain in the ‘dispersed’ formation (FIG. 2A).

To characterize the pinwheel effect in the presence of DNA and protein, and provide evidence of a polymer size-dependence on pinwheel formation, the following experiments were conducted. Using commercially-available silica-coated, iron-cored magnetic beads added to a microfluidic chamber in 4 to 8 M guanidine hydrochloride, conditions for driving nucleic acids to bind the silica surface, the RMF circulates the beads freely in a manner that has them reasonably distributed (FIG. 2A). The dispersed formation is stable and reproducible upon addition of 10 mg/mL bovine serum albumin (FIG. 2C), representing a 1000-fold excess mass of protein. However, a distinct transition to the ‘pinwheel’ formation was observed upon addition of nanogram levels of human genomic DNA (hgDNA), even with protein present (FIGS. 2D and 2B, respectively). This indicates that protein, even at excessively high concentrations, does not interfere with nucleic acid-induced pinwheel formation,

FIG. 3 shows a dynamic range of hgDNA-induced pinwheel formation over three orders of magnitude, from 10 ng/μL to 10 pg/μL. The mass of beads in the chamber was tuned to match the mass of hgDNA needed for pinwheel formation.

To further support the premise that DNA is the only analyte causing pinwheel formation under chaotropic salt conditions, sheared and unsheared hgDNA were evaluated. FIG. 4 shows that, for example, while extracted hgDNA resulted in pinwheel formation (FIG. 4A), the same mass of sonicated DNA (FIG. 4B) was similar to the negative control (dispersed) (FIG. 4C). Interestingly, FIG. 5 shows pinwheel formation is not exclusive to DNA or chaotropic conditions. Chitosan, a cationic polysaccharide (MW about 310 kDa), formed distinct pinwheels with the very same silica beads in a low-salt buffer (50 mM MES [2-(N-morpholino)ethanesulfonic acid] at pH 5). Here the binding is governed by electrostatic attraction, demonstrating that this detection method can be extrapolated with a different binding chemistry. This supports the position that this effect is a general phenomenon applicable to a wide variety of polymeric analytes.

The system described above provides a versatile, visual detection technique and related apparatus to detect and quantify polymeric molecules that bind to magnetic beads under certain conditions, e.g., conditions related to binding chemistries. Moreover, the technique may be conducted with only a minute mass of magnetic beads, as low as a few beads per assay, in a microfluidic chamber.

### Example II

### Exemplary Materials and Methods

Magnetic beads: MagneSil paramagnetic particle purchased from Promega Corporation, diameter=8±4 μm.

PMMA array: 4×4 array made by laser engraver, diameter of each well=0.2 in, capacity of each well=20 μL

Camera: Canon EOS Rebel XS

Microscope: Leica S8 APO

Stir plate: Thermix Stirrer Model 120S purchased from Fisher Scientific, Inc.

### Exemplary Procedure

1. Prepare GuHCl solution in 1×TE buffer with a concentration of 8 M. Concentrations of from about 100 mM to about 8 M may be employed. Other concentrations of guanidine hydrochloride, and other chaotropic salts, may be employed to drive nucleic acid to bind magnetic particles, such as magnetic particles having diameters disclosed herein. Moreover, different concentrations of salts may result in enhanced aggregation with certain diameters of magnetic beads, e.g., lower concentration of salts may result in enhanced aggregation of smaller diameter magnetic beads.

2. Prepare suspension of magnetic beads: take 30 μL of stock beads suspension, wash with water and GuHCl solution and resuspend in 1 mL GuHCl solution.

3. Prepare DNA sample:
a. Pre-purified DNA: dilute using 8 M GuHCl solution to appropriate concentrations
b. Cells or blood: mix cells or blood with copious 8 M GuHCl (e.g., volume ratio=1:100) to ensure cells are lysed and all the DNA is released.

4. Use DNA with a known concentration and with the same size of unknown DNA as standard, and prepare standard DNA solutions by serial dilution.

5. Mix a certain number of beads (e.g., 2-15 μL of suspension, depending on desired detection limit, sensitivity, and dynamic range) and a certain volume of standard DNA solutions (typically 5 μL) in the wells of PMMA plate. Adjust the total volume to 20 μL and GuHCl concentration to 6 M using GuHCl and/or H₂O.

6. Repeat step 5 for unknown DNA samples. With the PMMA plate, up to 16 DNA-magnetic beads mixtures can be prepared and measured together.

7. Put the PMMA array on stir plate and turn on the stir plate to mix the beads and DNA until the mixture system reaches equilibrium (about 5 minutes).

8. Adjust the PMMA array position on the stir plate so that one of the wells is at the center of stir plate. Turn on the stir plate to disperse beads in the centered well and take pictures.

9. Repeat step 8 for all the other wells containing samples.

10. Collect 5 pictures for each well.

11. Analyze pictures using ImageJ (see image processing).

12. Normalize the dark area values acquired from ImageJ by the area of dispersed beads without DNA, and plot the area percentage versus concentration of DNA.

### Exemplary Image Processing

Software: ImageJ v1,41 (Rasband, W. S., ImageJ, U.S. National Institutes of Health, Bethesda, Md., USA, http://rsb.info.nih.gov/ij/, 1997-2009), with multithresholder plugin (http://rsbweb.nih.gov/ij/plugins/multi-thresholder.html, Nov. 2, 2009).

Open 8-bit images, set threshold using triangle method in the multithresholder, click analyze->analyze particle to acquire the number of pixels below the threshold since beads are darker than background.

In triangle algorithm, the software sets the value of grey level that gives the maximum distance as shown below to be the threshold, (Zack et al., J. Histochem. Cytochem., 25:741 (1977)).

### Results

FIG. 10 shows the results of 5 and 10 μL of MagneSil paramagnetic particle suspension mixed with different amounts of HeLa cells. The graph is based on the assumption that there was 6.25 pg of DNA per cell.

### Example III

### Hybridization Induced Aggregation

### Methods

Into each well: 17 of 1×PCR buffer

1 μL of sample (suspected of having a specific target sequence). The sample may be heated using a heated stir plate at max RPM. covering the wall with a piece of glass to prevent evaporation, after which the following arc added:

1 μL of 5′ primer (oligonucleolide) containing beads

1 μL of 3′ primer (oligonucleotide) containing beads

A pinwheel forms in the center of the well when the complementary connector anneals to primer sequences and RMF is applied, which brings the beads together, then a picture is taken.

A. A 100 bp connection was formed when a connector (target) sequence 5′-AAATACGCCTCGAGTGCAGCCCATTT-3′ (SEQ ID NO:3) was mixed with beads having 5′-[BioTEG]TTTTTTATGTGGTCTATGTCGTCGTTCGCTAGTAGTTCCTGGG CTGCAC-3′ (SED ID NO:1) and 5′-TCGAGGCGTAGAATTCCCCCGATGCGCGCTGTICTIACTCATTTTT[Bio TEG-Q]-3 (SEQ ID NO:2), and that mixture subjected to an annealing temperature of 25° C. FIG. 11 shows the results obtained. The size of the pinwheel did not change with concentration, just the amount of pinwheels formed. Thus, the hybridization induced aggregation method can not only quantify the amount of connection but also can give a range of length of connection.
B. To detect a λ-DNA PCR product, a different working temperature was employed (70° C.). Primer Lambda_probe_{—}3′-CCAGTTGTACGAACACGAACTCATCTTTTTT[BioTEG-Q] (SEQ ID NO:4) Lambda_probe_{—}5′-[BioTEG]TTTTTTGGTTATCGAAATCAGCCACAGCGCC (SEQ ID NO:5) were employed to detect a 500 by PCR product (GATGAGTTCGTGITCGTACAACTGGCGTAATCATGGCCCTICGGGGC CATTGTTTCTCTGTGGAGGAGTCCATGACGAAAGATGAACTGATTGC CCGTCTCCGCTCGCTGGGTGAACAACTGAACCGTGATGTCAGCCTGA CGGGGACGAAAGAAGAACTGGCGCTCCGTGTGGCAGAGCTGAAAGA GGAGCTTGATGACACGGATGAAACTGCCGGTCAGGACACCCCTCTCA GCCGGGAAAATGTGCTGACCGGACATGAAAATGAGGTGGGATCAGC GCAGCCGGATACCGTGATTCTGGATACGTCTGAACTGGTCACGGTCG TGGCACTGGTGAAGCTGCATACTGATGCACTTCACGCCACGCGGGAT GAACCTGTGGCATTTGTGCTGCCGGGAACGGCGTTTCGTGTCTCTGCC GGTGTGGCAGCCGAAATGACAGAGCGCGGCCTGGCCAGAATGCAAT AACGGGAGGCGCTGTGGCTGATTTCGATAACC; SEQ ID NO:6).

However, a longer sequence (full length λ genomic DNA) had no effect, thus demonstrating specificity. The pinwheel size was different from that in A (above) due to the longer length of sequence between beads that were connected. via hybridization, resulting in a pinwheel that is less tight (compact) and so it appears larger.

C. Primer sequences typically used for qPCR are bound to a silica-like beads through streptavidin-biotin linkages. Beads having oligonucleotides with those linkages were prepared; forward primer: CGGGAAGGGAACAGGAGTAAG (SEQ NO:7); and reverse primer: CCAATCCCAGGTCTTCTGAACA (SEQ ID NO:8) Those sequences are specific for a 68 bp target region of a human TPDX locus (cgggaagggaacaagagtaagAccagcgcacagcccaacttgTgttcagaagacctgggattgg; SEQ ID NO:9). Pinwheels formed upon addition of hgDNA. For some hybridization induced aggregation assays, restriction enzymes or other nucleases may be employed to create smaller hgDNA fragments.

### Exemplary Applications for Hybridization Induced Aggregation Assays

The hybridization induced aggregation assay may be employed to detect specific DNAs in complex matrices, e.g., whole blood, DNAs such as cancer biomarkers, species specific DNA, e.g., human vs. animal detection in an unknown sample, male versus female detection or in an unknown sample, or exclusion of a suspect's DNA in criminal investigations. The assay allows for fluorescent label-free detection of specific sequences, is rapid (5 minutes) and is low cost, e.g., due to minimal instrumentation. The assay can be used to determine specific sequences of varying length and annealing temperatures, and so is a format suitable for multiplexing.

For example, a homogenous assay may be created where multiple targets can be detected simultaneously by simply ramping through a select temperature range while the oligonucleotides for different targets have disparate annealing temperature. Further, detection can occur even in the presence of competitive inhibition from an overwhelming mass of the competing DNA strand (about 1000 copies, or about 79000 pg/μL).

This method amplifies the light scattering by using larger particles, so a single binding event scatters more light than using 10-100 nm diameter gold nanoparticles. Thus, even larger particles that remain in suspension may be used for a lower optical limit of detection (LOD), so long as the linker can make the connection on the time scale of the collisions and strong enough to maintain that linkage.

### Example IV

### Detection and Quantification of Nucleated Cells

To determine if cell number can be quantitated using the pinwheel assay, magnetic particles with DNA (concentrations may be at least 1 to 5 pg/uL, assuming 6.25 pg/cell) and without DNA from whole blood were subjected to RMF. As discussed above, detection of aggregates may be accomplished using a camera, a light source, a rotating magnetic field (RMIT), a substrate for the sample such as PDMS-glass microwell chip, and magnetic particles, e.g., magnetic beads such as super paramagnetic silica-coated particles (about 5 μm in diameter).

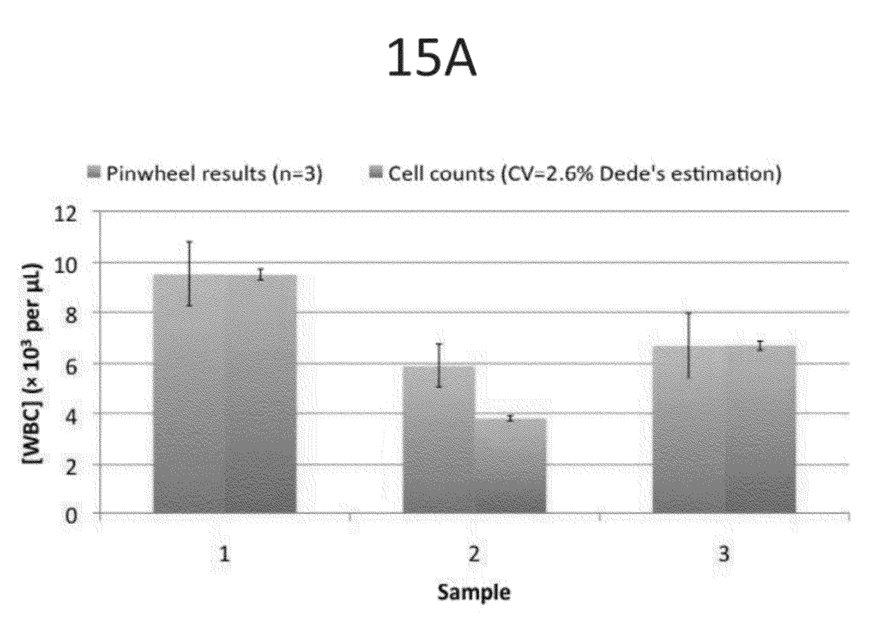

Approximately 5 photographs are taken over 30 seconds after RMF is applied. For image analysis, a threshold value is set automatically by isodata algorithm. Pixels below threshold are considered dark, and the dark area of a sample without DNA is used to normalize DNA sample data. The dark area of a sample with DNA over the dark area of a sample without DNA times 100 is the dark area percent (%) see FIG. 14).

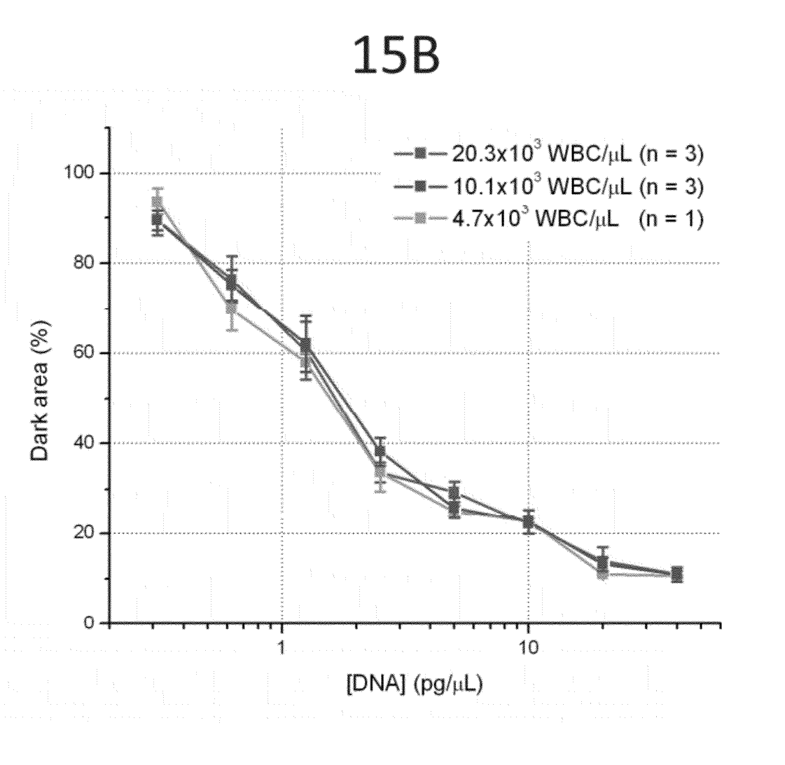

FIG. 15 show a comparison of the use of aggregates versus a Coulter Counter to determine white blood cell counts in three samples. The results demonstrated that the pinwheel effect can be utilized to define the concentration of DNA directly from blood samples, and that different concentrations for each correlated with measurement of DNA via the conventional method. The results show that the pinwheel assay can be used to determine cell number.

FIG. 15B shows the result of diluting each sample to equalize the number of nucleated cells per microliter in each sample. A pinwheel assay of the diluted samples yielded overlapping curves, certifying that a consistent pinwheel response was obtained and that the degree of bead aggregation tracked with the number of WBC in each sample.

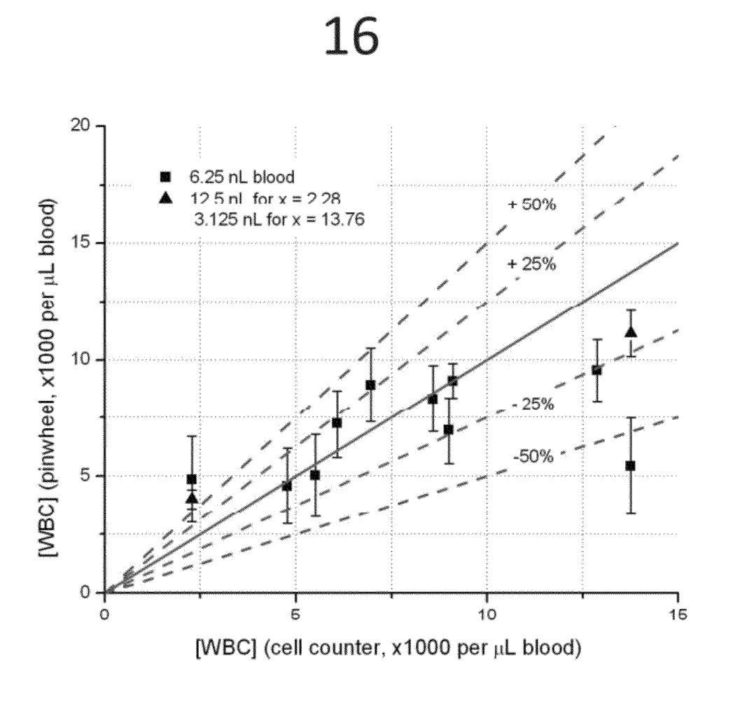

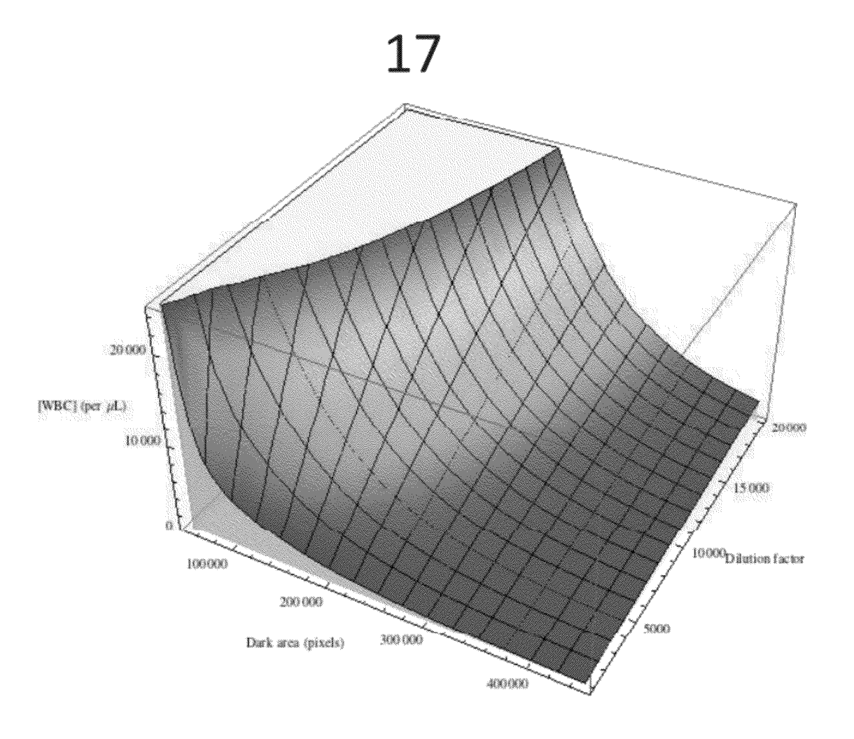

FIG. 16 shows a direct comparison between the pinwheel assay and a Coulter Counter assay. The results for the pinwheel assay correlate quite well with those from the Coulter Counter. The data points on the red solid line represent pinwheel results that equal that of a cell counter. Points between the two red dashed lines represent the results with <25% errors, and the blue lines represent mean 50% error. WBC counts between 5,000 and 10,000 per μL of blood were generally within 25% error, while outside of that range, >50% error was observed. This may be corrected by dilution of the blood sample (see FIG. 17). Thus, given an unknown blood sample, once could dilute it to a certain fold, and acquire a dark area value from the pinwheel assay. Based on a standard such as the one in panel A of FIG. 17, the concentration of WBCs may be plotted as a function of dark area and the dilution factor, from which the concentration of WBC can be read directly after a pinwheel assay.

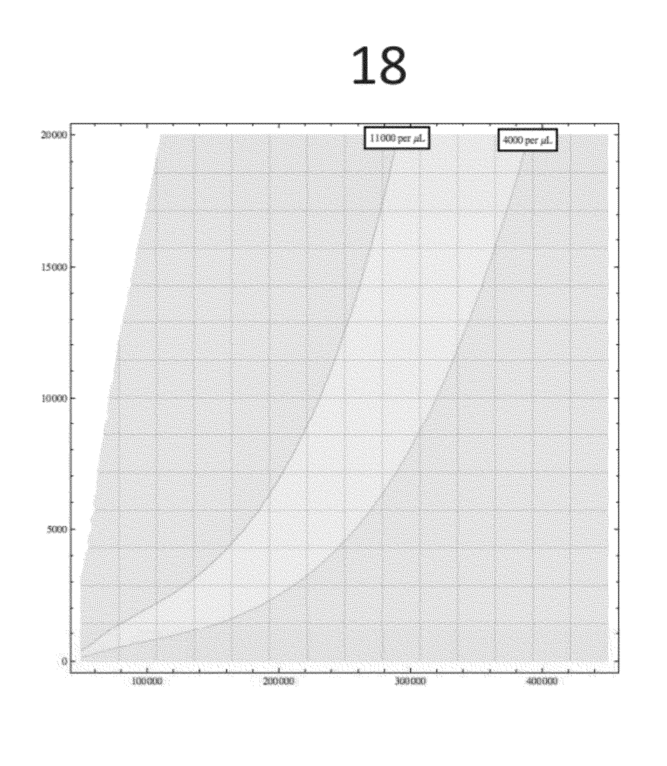

FIG. 18 is a simplified 2D version of FIG. 17. For example, one could dilute an unknown sample to a certain fold, obtain the dark area, and find its position in FIG. 18. Thus, the method can be used to test if the concentration of WBCs in an unknown blood sample is within the normal range (4,000 to 11,000 per μL).

The following parameters were used for cell quantification.

```
SetDirectory[“Desktop/Pinwheel X/2011_02_21_M13 samples”];
  func1[histodata_,threshold_]:=
    Module[{mean1,mean2,thr,greylevel,i},
      greylevel=Table[i,{i,256}];
If[Total[Take[histodata,threshold]]==0,mean1=0,mean1=Round[Total
[Take[greylevel*histodata,threshold]]/Total[Take[histodata,threshold]]]];
      If[Total[Take[histodata,threshold-
256]]==0,mean2=0,mean2=Round[Total[Take[greylevel*histodata,
threshold-
256]]/Total[Take[histodata,threshold-256]]]];
      thr=Round[(mean1+mean2)/2]
      ];
  func2[filenames_]:=
    Module[{data1,data2,data3,threshold,darkarea,a},
      data1=ImageData[ImageResize[Import[filenames],Scaled[1/10]]];
      data2=data1 //. {a_,b_,c_}->a;
      data3=BinCounts[Flatten[data2],{0,1,1/256}];
      threshold=FixedPoint[func1[data3,#]&,128];
      darkarea=Total[Take[data3,threshold]]
      ];
  filenames=FileNames[“*.JPG”];
filenumber=Total[Dimensions[filenames]];
results=Table[func2[filenames[[i]]],{i,filenumber}];
Export[DateString[{“Year”,“_”,“Month”,“_”,“Day”,“_”,“Hour”,“Minute”,
“Second”,“_”}]<>“result.xls”,{Transpose[Join[{filenames},{results}]]}];
```

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method for quantitatively detecting the number of DNA containing cells in a biological sample, comprising:
a) providing a sample suspected of comprising cells having genomic DNA;
b) contacting the sample with magnetic beads under conditions that allow for binding of DNA from the cells to the beads so as to form a mixture;
c) subjecting the mixture to an amount of energy; and
d) detecting the amount of aggregate formation by the beads subjected to the energy, thereby quantifying the number of cells in the sample.

2. The method of claim 1 wherein the mixture is subjected to a rotating magnetic field, acoustic energy or vibration.

3. The method of claim 2 wherein the magnetic field is generated by a magnet.

4. The method of claim 1 wherein pinwheel formation of the aggregates is detected.

5. The method of claim 1 wherein the sample is subjected to sonication, shearing or a nuclease.

6. The method of claim 1 wherein the sample comprises lysed cells.

7. The method of claim 6 wherein the sample comprises a subfraction of the lysed cells.

8. The method of claim 1 wherein the sample comprises amplified DNA.

9. The method of claim 1 wherein the sample is a physiological fluid sample.

10. The method of claim 9 wherein the physiological fluid sample is a urine sample or a cerebrospinal fluid sample.

11. The method of claim 1 wherein the sample is a blood sample.

12. The method of claim 1 wherein the sample comprises human cells.

13. The method of claim 1 wherein the sample is a tissue biopsy.

14. The method of claim 1 wherein the sample is subjected to cell size selection prior to contacting.

15. The method of claim 1 wherein the sample is subjected to antibody-based. selection of a subpopulation of cells in the sample,

16. The method of claim 1 wherein the magnetic beads are coated or derivatized with silica, amine-based charge switch, boronic acid, silane, oligonucleotides, lectins, PNA, LNA, antibody, antigen, avidin or biotin.

17. The method of claim 1 wherein the magnetic beads further comprise a fluorescent label.

18. The method of claim 1 wherein the contacting of the sample with the beads is in the presence of concentrated chaotropic salts.

19. The method of claim 1 wherein the mixture is in a detection chamber that forms part of a microfluidic device.

20. The method of claim 2 wherein the magnetic field is generated by a U-shaped magnet.


SEQUENCE LISTING (10 sequences)

SEQ ID NO: 1 - DNA, 49 bp, Artificial Sequence
  A synthetic primer
ttttttatgtggtctatgtcgtcgttcgctagtagttcctgggctgcac

SEQ ID NO: 2 - DNA, 46 bp, Artificial Sequence
  A synthetic primer
tcgaggcgtagaattcccccgatgcgcgctgttcttactcattttt

SEQ ID NO: 3 - DNA, 26 bp, Artificial Sequence
  A connector (target sequence)
aaatacgcctcgagtgcagcccattt

SEQ ID NO: 4 - DNA, 31 bp, Artificial Sequence
  A synthetic primer
ccagttgtacgaacacgaactcatctttttt

SEQ ID NO: 5 - DNA, 31 bp, Artificial Sequence
  A synthetic primer
ttttttggttatcgaaatcagccacagcgcc

SEQ ID NO: 6 - DNA, 500 bp, Homo sapien
gatgagttcgtgttcgtacaactggcgtaatcatggcccttcggggccattgtttctctgtggaggagtccatgacgaaagatgaactgattgcccgtctccgctcgctgggtgaacaactgaaccgtgatgtcagcctgacggggacgaaagaagaactggcgctccgtgtggcagagctgaaagaggagcttgatgacacggatgaaactgccggtcaggacacccctctcagccgggaaaatgtgctgaccggacatgaaaatgaggtgggatcagcgcagccggataccgtgattctggatacgtctgaactggtcacggtcgtggcactggtgaagctgcatactgatgcacttcacgccacgcgggatgaacctgtggcatttgtgctgccgggaacggcgtttcgtgtctctgccggtgtggcagccgaaatgacagagcgcggcctggccagaatgcaataacgggaggcgctgtggctgatttcgataacc

SEQ ID NO: 7 - DNA, 21 bp, Artificial Sequence
  A synthetic primer
cgggaagggaacaggagtaag

SEQ ID NO: 8 - DNA, 22 bp, Artificial Sequence
  A synthetic primer
ccaatcccaggtcttctgaaca

SEQ ID NO: 9 - DNA, 64 bp, Homo sapien
cgggaagggaacaggagtaagaccagcgcacagcccgacttgtgttcagaagacctgggattgg

SEQ ID NO: 10 - DNA, 20 bp, Artificial Sequence
  A synthetic primer
atgcggagctcacgtcgggt